(19) United States
(12) Patent Application Publication
(10) Pub. No.: US 2010/0233676 A1
(43) Pub. Date: Sep. 16, 2010

(54) HIGH AFFINITY FLUOROCHROME BINDING PEPTIDES

(76) Inventors: Kimberly Kelly, Burlington, MA (US); Ralph Weissleder, Peabody, MA (US)

Correspondence Address: FISH & RICHARDSON PC, P.O. BOX 1022, MINNEAPOLIS, MN, 55440-1022, US

(21) Appl. No.: 12/663,370

(22) PCT Filed: Jun. 12, 2008
(86) PCT No.: PCT/US08/07329

§ 371 Date: May 25, 2010

Related U.S. Application Data
(60) Provisional application No. 60/934,558, filed on Jun. 14, 2007.

(51) Int. Cl. C12Q 1/04; C07K 5/10; C07K 7/06; C07K 7/08; C07K 14/00; C12Q 1/70; C12N 15/63; C12N 15/85; C12N 15/74; C12N 15/82

(52) U.S. Cl. 435/5; 530/330; 530/329; 530/328; 530/327; 530/326; 530/402; 435/34; 435/440; 435/455; 435/471; 435/468

(57) ABSTRACT

The present invention contemplates strategies comprising small molecule, cell permeable probes that allow site-specific protein labeling for visualizing biological processes. In one embodiment, the present invention contemplates a series of short peptide sequences comprising high affinity binding (i.e., for example, subnanomolar affinity (0.53 nM) for indocyanine fluorochromes. In one embodiment, the peptide sequences comprise a 5 pmol detection limit for indocyanine fluorochromes. In one embodiment, the present invention contemplates methods comprising high affinity peptide-fluorochrome binding pairs in biological applications including, but not limited to, enzyme linked immunoabsorbent assay (ELISA), fluorescence activated cell sorting (FACS), microscopy (i.e., for example scanning electromicroscopy), Western Blots, histochemistry, protein and cell based tracking both in vitro and in vivo.

### STATEMENT OF GOVERNMENT INTEREST

The present invention was partially supported by National Cancer Institute Grant No. 2-P50-CA083655-06. The US government, therefore, has certain rights and interests in the present invention.

### FIELD OF INVENTION

The present invention is related to compositions and methods to identify and use unique peptide sequences that interact with small molecule imaging agents. The invention utilizes alternative phage libraries conjugated to imaging agents for screening cyclic and linear peptide sequences that bind with high affinity. For example, the peptide sequences comprise submicromolar affinities for the imaging agents. In one embodiment, the present invention contemplates a composition comprising a high affinity peptide/imaging agent binding pair that will be useful in the diagnosis and treatment of medical conditions.

### BACKGROUND

A number of advances in reporter and imaging technologies have led to the widespread use of fluorescent proteins and site-specific binders. Chimeric fluorescent reporter proteins (FPs) that are fusions of a targeting protein and green fluorescent protein (GFP) variants can often provide spatio-temporal information and images from diverse cellular processes. (i.e., for example, mitosis, DNA repair, cytoskeletal remodeling, receptor trafficking, focal adhesion, local calcium sensing, membrane potential, pH sensing and microbial pathogenesis. Panchal et al., Proc Natl Acad Sci USA, 100:15936-15941 (2003); Giepmans et al., 312:217-224 (2006); and Chen et al., Nat Methods, 2:99-104 (2005). Fusion proteins have the potential to demonstrate one-on-one targeting, but they are constrained by stability, fluorescence, and sensor properties of the specific GFP. Consequently, a broad applicability as nanoscale indicators remains to be demonstrated. In addition, steric hindrance FP problems when in proximity to a target disrupt the native interactions of nearby proteins in multimeric complexes and/or alter localization. Marguet et al., Immunity, 11:231-40 (1999); and Lisenbee et al., Traffic 4:491-501 (2003).

Alternatives to FP commonly use small molecule site specific labeling strategies. Recently developed methods have employed special peptide handles that: i) recruit small molecule ligands (Farinas et al., J Biol Chem, 274:7603-7606 (1999)); ii) harness enzyme activity to catalyze conjugation of tags (Tannous et al., Nat Methods, 3:391-396 (2006)); and iii) use cellular protein machinery (Keppler et al., Proc Natl Acad Sci USA, 101:9955-9959 (2004). Nevertheless, some of these methods suffer from disadvantages regarding low affinity, low signal-to-noise ratios and multi-step reactions that rely on exogenous enzymes. Stroffekova et al., Pflugers Arch, 442, 859-866 (2001); Chen et al., Curr Opin Biotechnol, 16:35-40 (2005).

What is needed are biological ligands tailored specifically to a tag, wherein the ligands have affinity constants surpassing those of monoclonal antibodies.

### SUMMARY

The present invention is related to compositions and methods to identify and use unique peptide sequences that interact with small molecule imaging agents. The invention utilizes alternative phage libraries conjugated to imaging agents for screening cyclic and linear peptide sequences that bind with high affinity. For example, the peptide sequences comprise submicromolar affinities for the imaging agents. In one embodiment, the present invention contemplates a composition comprising a high affinity peptide/imaging agent binding pair that will be useful in the diagnosis and treatment of medical conditions.

In one embodiment, the present invention contemplates a peptide comprising a high affinity binding site to a benzindol fluorochrome. In one embodiment, the high affinity binding site comprises a submicromolar affinity to said fluorochrome. In one embodiment, the high affinity binding site comprises an affinity of less than 0.1 nanomolar to the fluorochrome. In one embodiment, the high affinity binding site comprises an affinity of approximately between 10-100 picomolar to the fluorochrome. In one embodiment, the high affinity binding site comprises an affinity of approximately between approximately 1-10 picomolar to the fluorochrome. In one embodiment, the high affinity binding site comprises between approximately four to fifteen amino acids. In one embodiment, the binding site comprises I, S, and at least two F's. In one embodiment, the at least two F's are consecutive. In one embodiment, the binding site further comprises Q. In one embodiment, the binding site further comprises P. In one embodiment, the binding site further comprises H. In one embodiment, the P and H are consecutive. In one embodiment, the binding site comprises IQSPHFF. In one embodiment, the binding site comprises IQSPHFFGGSK In one embodiment, the fluorochrome is selected from the group comprising VT680, GH680, AF750, Cy3.5 or Cy5.5. In one embodiment, the peptide is part of a fusion protein. In one embodiment, the fusion protein further comprises a cell targeting moiety. In one embodiment, the cell targeting moiety comprises platelet derived growth factor receptor.

In one embodiment, the present invention contemplates a vector comprising a promoter operably linked to a nucleic acid sequence encoding a peptide, wherein the peptide comprises a high affinity binding site to a benzindol fluorochrome. In one embodiment, the high affinity binding site comprises a submicromolar affinity to said fluorochrome. In one embodiment, the high affinity binding site comprises an affinity of less than 0.1 nanomolar to the fluorochrome. In one embodiment, the high affinity binding site comprises an affinity of approximately between 10-100 picomolar to the fluorochrome. In one embodiment, the high affinity binding site comprises an affinity of approximately between approximately 1-10 picomolar to the fluorochrome. In one embodiment, the high affinity binding site comprises between approximately four to fifteen amino acids. In one embodiment, the binding site comprises I, S, and at least two F's. In one embodiment, the at least two F's are consecutive. In one embodiment, the binding site further comprises Q. In one embodiment, the binding site further comprises P. In one embodiment, the binding site further comprises H. In one embodiment, the P and H are consecutive. In one embodiment, the binding site comprises IQSPHFF. In one embodiment, the binding site comprises IQSPHFFGGSK. In one embodiment, the fluorochrome is selected from the group comprising VT680, GH680, AF750, Cy3.5 or Cy5.5.

In one embodiment, the present invention contemplates a transgenic animal whose genome comprises a transgene encoding a peptide comprising a high affinity binding site to a fluorochrome. In one embodiment, the fluorochrome comprises a benzindol fluorochrome. In one embodiment, the transgenic animal further comprises a diseased tissue. In one embodiment, the genome is derived from the diseased tissue. In one embodiment, the transgene is stably integrated into the genome of the transgenic animal. In one embodiment, the peptide is expressed by the transgene. In one embodiment, the peptide is displayed on the surface of the diseased tissue. In one embodiment, the peptide comprises the amino acid sequence IQSPHFF (SEQ ID NO:1). In one embodiment, the peptide comprises the amino acid sequence HHSHRHH (SEQ ID NO: 16).

In one embodiment, the present invention contemplates a method, comprising: a) providing; i) a benzindol fluorochrome; and ii) a host capable of displaying a peptide comprising a high affinity binding site to the benzindol fluorochrome; b) displaying the peptide on the host; and c) contacting the displayed peptide with the fluorochrome under conditions such that the host is detectable. In one embodiment, the host comprises a living cell. In one embodiment, the living cell is derived from a cell line. In one embodiment, the living cell comprises a bacterial cell. In one embodiment, the living cell comprises a cell line. In one embodiment, the cell line is selected from the group consisting of HEK-293T cells, HT1080 cells, HeLa cells, Daudi cells, K562 cells, or COS cells. In one embodiment, the living cell is derived from a subject. In one embodiment, the subject comprises a mammal. In one embodiment, the mammal is a human mammal. In one embodiment, the mammal is a non-human mammal. In one embodiment, the living cell comprises a plant cell. In one embodiment, the host comprises a virus. In one embodiment, the high affinity binding site comprises a submicromolar affinity to said fluorochrome. In one embodiment, the high affinity binding site comprises an affinity of less than 0.1 nanomolar to the fluorochrome. In one embodiment, the high affinity binding site comprises an affinity of approximately between 10-100 picomolar to the fluorochrome. In one embodiment, the high affinity binding site comprises an affinity of approximately between approximately 1-10 picomolar to the fluorochrome. In one embodiment, the high affinity binding site comprises between approximately four to fifteen amino acids. In one embodiment, the binding site comprises I, S, and at least two F's. In one embodiment, the at least two F's are consecutive. In one embodiment, the binding site further comprises Q. In one embodiment, the binding site further comprises P. In one embodiment, the binding site further comprises H. In one embodiment, the P and H are consecutive. In one embodiment, the binding site comprises IQSPHFF. In one embodiment, the binding site comprises IQSPHFFSSGK. In one embodiment, the fluorochrome is selected from the group comprising VT680, GH680, AF750, Cy3.5 or Cy5.5.

In one embodiment, the present invention contemplates a method, comprising: a) providing; i) a vector comprising a promoter operably linked to a nucleic acid sequence encoding a peptide, wherein the peptide comprises a high affinity binding site to a benzindol fluorochrome; ii) a host, wherein said host is capable of undergoing transformation by said vector; b) transfecting said host with said vector under conditions such that said amino acid sequence is expressed. In one embodiment, the host comprises a living cell. In one embodiment, the living cell comprises a bacterial cell. In one embodiment, the living cell is derived from a cell line. In one embodiment, the cell line is selected from the group consisting of HEK-293T cells, HT1080 cells, HeLa cells, Daudi cells, K562 cells, or COS cells. In one embodiment, the living cell is derived from a subject. In one embodiment, the subject comprises a mammal. In one embodiment, the mammal is a human mammal. In one embodiment, the mammal is a non-human mammal. In one embodiment, the living cell comprises a plant cell. In one embodiment, the host comprises a virus. In one embodiment, the promoter is selected from the group comprising a CMV promoter, a SV40 promoter, a metallothionein promoter, a murine mammary tumor virus promoter, a Rous sarcoma virus promoter, or a polyhedrin promoter. In one embodiment, the high affinity binding site comprises a submicromolar affinity to said fluorochrome. In one embodiment, the high affinity binding site comprises an affinity of less than 0.1 nanomolar to the fluorochrome. In one embodiment, the high affinity binding site comprises an affinity of approximately between 10-100 picomolar to the fluorochrome. In one embodiment, the high affinity binding site comprises an affinity of approximately between approximately 1-10 picomolar to the fluorochrome. In one embodiment, the high affinity binding site comprises between approximately four to fifteen amino acids. In one embodiment, the binding site comprises I, S, and at least two F's. In one embodiment, the at least two F's are consecutive. In one embodiment, the binding site further comprises Q. In one embodiment, the binding site further comprises P. In one embodiment, the binding site further comprises H. In one embodiment, the P and H are consecutive. In one embodiment, the binding site comprises IQSPHFF. In one embodiment, the fluorochrome is selected from the group comprising VT680, GH680, AF750, Cy3.5 or Cy5.5.

In one embodiment, the present invention contemplates a method of making a transgenic animal, comprising: a) providing; i) a vector comprising a promoter operably linked to a nucleic acid sequence encoding a peptide, wherein the peptide comprises a high affinity binding site to a benzindol fluorochrome; ii) an animal comprising a tissue having at least one cell, wherein the cell is capable of undergoing stable transfection by the vector; and b) contacting the vector with the cell under conditions such that the cell becomes transfected with the vector. In one embodiment, the transfection results in a stable integration of the vector. In one embodiment, the promoter is selected from the group comprising a CMV promoter, a SV40 promoter, a metallothionein promoter, a murine mammary tumor virus promoter, a Rous sarcoma virus promoter, or a polyhedrin promoter.

In one embodiment, the present invention contemplates a method, comprising: a) contacting a sample with a small high affinity peptide/fluorochrome binding pair, wherein the sample is derived from a subject administered a biological agent; b) incubating the sample with the binding pair; c) illuminating the sample with light of a wavelength absorbable by the binding pair; d) detecting a signal emitted from the binding pair; e) forming an optical image from the emitted signal; f) read ministering the agent to the subject and repeating steps (a)-(e); and (g) comparing the emitted signals and images of steps (d) and (e) over time or at different agent doses to assess the activity of the agent. The sample can include, but is not limited to, cells, cell culture, tissue section, cytospin samples, or the like. In one embodiment, the agent can be a therapeutic compound. In one embodiment, the therapeutic compound can be an enzyme inhibitor selected from the group comprising a proteinase inhibitor, a kinase inhibitor, a transferase inhibitor, or a polymerase inhibitor including, but not limited to, upstream transcription regulators. In one embodiment, the method further comprises identifying the efficacy of an agent (i.e., for example, a therapeutic drug candidate). In one embodiment, the method further comprises assessing agent levels in a subject (i.e., for example, intracellular tissue levels and/or blood, plasma, or serum levels). In one embodiment, the method further comprises, optimizing an agent therapy, wherein the dose, timing and/or administration route of a given therapeutic agent is optimized. In one embodiment, the method is performed at least twice, once with and once without, administering to the subject the agent, thereby providing a comparison of the outcome of the two methods for assessing the activity of the agent. The methods may also be performed prior to administration of the agent to determine whether a target (i.e., for example, a drug target) is present and/or expressed, and therefore whether the agent should be administered to the subject. In one embodiment, the administration of the agent to the subject is performed throughout the method including, without limitation, prior to administering the binding pair. In one embodiment, a portion of the binding probe is detected by other means, including, but not limited to, a second fluorescent wavelength, bioluminescence, changes in magnetic properties, and/or gamma radiation. In one embodiment, a second binding pair is administered to determine a local concentration of the first binding pair.

The invention also includes a method for determining the presence of a composition (e.g., a drug or a polypeptide expressed by a gene, such as a gene introduced into the subject by gene therapy techniques) in a subject. The methods of the present invention may further be used for high throughput testing of therapeutic drug candidates (e.g., combinatorially designed therapeutic drug candidates). The methods can also be used to select drug candidates for clinical testing.

In one embodiment, the present invention contemplates an in vivo optical imaging method for guiding therapeutic, e.g., surgical, interventions by: (a) administering to a subject a small high affinity peptide/fluorochrome binding pair; (b) illuminating a target tissue with light of a wavelength absorbable by the binding pair; and (c) detecting an optical signal emitted by the binding pair. In one embodiment, the subject comprises a mammal. In one embodiment, the mammal is a human. Although it is not necessary to understand the mechanism of an invention, it is believed that the method can be used to help a physician or surgeon to identify and characterize areas of disease, such as colon polyps or vulnerable plaque, to distinguish diseased and normal tissue, such as detecting tumor margins that are difficult to detect using an ordinary operating microscope. e.g., in brain surgery, and help dictate a therapeutic or surgical intervention, e.g., by determining whether a lesion is cancerous and should be removed or non-cancerous and left alone.

### DEFINITIONS

The term “chromophore” as used herein, includes, but is not limited to, a fluorochrome (i.e., for example, organic and/or inorganic), non-fluorochrome chromophore, fluorescence quencher, or absorption chromophore. Thus, for example, an imaging probe comprises a chromophore chemically linked to a high affinity (i.e., for example, submicromolar) small peptide. Alternatively, a chromophore may be substituted with, for example, a benzindol moiety. Benzindol fluorochromes may include, but are not limited to, VT680 (an —NHS ester), GH680 (VT680+ Genhance® dye), AF750, Cy3.5, or Cy 5.5.

The term “chemically linked”, as used herein, refers to any interaction by any attractive force between atoms strong enough to allow the combined aggregate to function as a unit. This includes, but is not limited to, chemical bonds such as covalent bonds (i.e., for example, polar, or nonpolar), and non-covalent bonds such as ionic bonds, metallic bonds, and/or bridge bonds.

The term “protective chain”, as used herein, refers to any biocompatible moiety covalently linked to a chromophore to inhibit undesired biodegradation, clearance, or immunogenicity of the probe. Suitable protective chains include, but are not limited to, polyethylene glycol, methoxypolyethylene glycol, methoxypolypropylene glycol, copolymers of polyethylene glycol and methoxypolypropylene glycol, polylactic-polyglycolic acid, poloxamer, polysorbate 20, dextran and its derivatives, starch and starch derivatives, and fatty acids and their derivatives. In some embodiments of the invention, the chromophore attachment moiety is polylysine and the protective chains are methoxypolyethylene glycol.

The term “targeting moiety” or “cell targeting moiety” as used herein, refers to any moiety bound covalently or noncovalently to a chromophore, which moiety enhances the concentration of the probe in a target tissue relative to surrounding tissue. For example, a fluorochrome may be bound to an antibody that enhances the concentration of the fluorochrome at a tumor.

The term “disease” as used herein, refers to any impairment of the normal state of the living animal or plant body or one of its parts that interrupts or modifies the performance of vital functions, that is typically manifested by distinguishing signs and symptoms. For example, a disease may include, but is not limited to, cancer diseases, cardiovascular diseases, neurodegenerative diseases, immunologic diseases, autoimmune diseases, inherited diseases, infectious diseases, bone diseases, and environmental diseases.

The term “host” as used herein, refers to any living cell or virus comprising homologous nucleic acids capable of incorporating heterologous nucleic acids. Such living cells may include, but are not limited to, mammalian cells, bacterial cells, or plant cells. Further, a living cell may be derived from a “subject”, thereby providing in vivo heterologous nucleic acid incorporation. Alternatively, a living cell may be derived from cell lines thereby providing in vitro heterologous nucleic acid incorporation. Cells lines may include, but are not limited to, HEK-293T cells, HT1080 cells, HeLa cells, Daudi cells, K562 cells, or COS cells.

The term “subject” as used herein, refers to a mammal, including a human, or a non-human animal. A non-human animal may represent a model of a particular disease or disorder (i.e., for example, a mouse, rat, rabbit etc). Alternatively, a non-human animal may represent a domesticated pet and/or livestock in need of treatment and/or diagnosis for a disease (i.e., for example, dog, cat, goat, bovine, ovine, etc.).

The term, “backbone”, as used herein, refers to any biocompatible polymer to which infrared and/or near infrared fluorochromes are covalently linked. For example, a polymer may include, but are not limited to, polypeptides or synthetic polymers.

The term, “binding site”, as used herein, refers to any portion of a first molecule to which a second molecule may attach. Such attachments include, but are not limited to, non-covalent (i.e., for example, ionic, Van der Waals forces, electrostatic, etc) or covalent.

The term, “high affinity”, as used herein, refers to any ligand that attaches to another molecule having a submicromolar K_{D}. For example, a high affinity ligand may have an affinity residing in the nanomolar range. Alternatively, a high affinity ligand may have an affinity residing in the picomolar range. As used in the present invention, the term, high affinity does not refer to generally accepted micromolar binding affinities of most known biological ligands (i.e., for example, antibodies, hormones, and naturally produced peptides).

The term, “binding pair” as used herein, refers to a specific and stable interaction between a first and second compound. Such an interaction may be stabilized by a high affinity binding site. Binding pair's may undergo either non-covalent (i.e., for example, temporary) or covalent (i.e., for example, permanent) bonding.

The term “promoter” as used herein, refers to any nucleic acid sequence that directs nucleic acid transcription. As used herein, a promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation.

The term “operably linked”, as used herein, refers to any functional linkage between a nucleic acid expression control sequence (i.e., for example, a promoter, or other array of transcription factor binding sites) and a second nucleic acid sequence, wherein the expression control sequence directs transcription of the nucleic acid corresponding to the second sequence.

The term “heterologous” as used herein, refers to any nucleic acid wherein two or more subsequences within the nucleic acid are not found in the same relationship to each other in nature. For example, a recombinant nucleic acid may typically have two or more sequences from unrelated genes arranged to make a new functional nucleic acid.

The term “isolated nucleic acid molecule” as used herein, refers to any nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

The term “nucleic acid” as used herein, refers to any deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

The term “amino acid” as used herein, refers to any naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, γ-carboxyglutamate, and O-phosphoserine, phosphothreonine.

The terms “protein”, “peptide” and “polypeptide” as used herein, describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes, but is not limited to, full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The term “selectively (or specifically) hybridizes to” as used herein, refers to any binding, duplexing, or hybridizing of a molecule only to a particular nucleotide sequence under stringent hybridization conditions when that sequence is present in a complex mixture (for example, total cellular or library DNA or RNA).

The term “stringent hybridization conditions” as used herein, refers any condition under which a probe will hybridize to its target complementary sequence, typically in a complex mixture of nucleic acids, but to no other sequences. Stringent conditions are sequence-dependent and circumstance-dependent; for example, longer sequences can hybridize with specificity at higher temperatures. In: Techniques in Biochemistry and Molecular Biology-Hybridization with Nucleic Probes, Tijssen et al., “Overview of principles of hybridization and the strategy of nucleic acid assays” (1993) (incorporated herein by reference in its entirety). For example, a “stringent hybridization condition” results in the stable hybridization of nucleotide sequences that are at least 60% homologous to each other. Preferably, the conditions are such that sequences at least about 65%, more preferably at least about 70%, and even more preferably at least about 75% or more homologous provide a stable hybridization of two nucleotide sequences.

Generally, stringent conditions are selected to be about 5 to 10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions also may be achieved with the addition of destabilizing agents, for example, formamide. For selective or specific hybridization, a positive signal is at least two times background, preferably 1× background hybridization.

Exemplary stringent hybridization conditions can be as following, for example: 50% formamide, 5×SSC and 1% SDS, incubating at 42° C., or 5×SSC and 1% SDS, incubating at 65° C., with wash in 0.2×SSC and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C. to 95° C. for 30 sec. to 2 min., an annealing phase lasting 30 sec. to 2 min., and an extension phase of about 72° C. for 1 to 2 min.

The term “moderately stringent hybridization conditions” as used herein, refers to any condition allowing hybridization of nucleic acids that do not hybridize to each other under stringent conditions but are still substantially identical (i.e., if the polypeptides which they encode are substantially identical). This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is a simple estimate and may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985)). Other references include more sophisticated computations which take structural as well as sequence characteristics into account for the calculation of Tₘ.

The term “amplifiable nucleic acid” is used in reference to nucleic acids which may be amplified by any amplification method. It is contemplated that “amplifiable nucleic acid” will usually comprise “sample template.”

The term “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

The term “sample template” as used herein, refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “amplification” as used herein, refers to any production of additional copies of a nucleic acid sequence and is generally carried out using polymerase chain reaction technologies. (Dieffenbach C W and G S Dveksler (1995) PCR Primer, a Laboratory Manual, Cold Spring Harbor Press, Plainview N.Y.).

The term “polymerase chain reaction” (“PCR”) as used herein, refers to the method of K. B. Mullis U.S. Pat. Nos. 4,683,195 and 4,683,202, hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

The term “reverse transcription polymerase chain reaction” or “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and of an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “about” or “approximately” as used herein, in the context of numerical values and ranges refers to values or ranges are close to the recited values or ranges such that the invention can perform as intended.

The term “vector” as used herein, refers to any nucleic acid sequence comprising a protein coding sequence of interest, a promoter, and a termination sequence wherein the vector is capable of expressing the protein coding sequence of interest after entering a host cell (i.e., transfection). A cell may be transiently transfected (vector linearization in the cytoplasm), or a cell may be stably transfected (vector insertion within the host cell genome).

The term “consecutive” as used herein, refers to at least two amino acid residues following one another in uninterrupted order.

The term “biological agent” as used herein, refers to any compound and/or molecule capable of having an effect on living tissue. For example, a biological agent may include, but not be limited to, a drug, a hormone, an antigen, an antibody, a peptide, or a nucleic acid (i.e., for example, an antisense). A biological agent may comprise a “therapeutic compound” wherein the primary purpose of the therapeutic compound is to improve symptoms of a specific disease or adverse medical condition.

The term “optical imaging” as used herein, refers to any device and or method resulting in graphical information regarding the biological state of a living tissue and/or cell. For example, optical imager may include, but are not limited to, positron emission tomography (PET) and single-photon emission tomography (SPECT), magnetic resonance (MR) imaging, or spectroscopy.

The term “disease” as used herein, refers to any disordered or incorrectly functioning organ, part, structure, or system of the body resulting from the effect of genetic or developmental errors, infection, poisons, nutritional deficiency or imbalance, toxicity, or unfavorable environmental factors; illness; sickness; or ailment.

The term “symptom” as used herein, refers to any phenomenon that arises from and accompanies a particular disease or disorder thereby serving as an indicator. The term “protease resistant”, as used herein, refers to any peptide or protein that comprise a tertiary or quaternary structure that interferes with protease attachment and subsequent degradation.

The term “protease”, as used herein, refers to any enzyme that catalyses the splitting of interior peptide bonds (i.e., for example, an endoprotease) or terminal peptide bonds (i.e., for example, an exoprotease) in a protein.

The term “protecting group”, as used herein, refers to any molecule that is added to a molecule for the purpose of providing degradation protection. For example, peptides or proteins may have protecting groups for protection against proteases. Protecting groups may include, but are not limited to, N-terminal acetyl groups and C-terminal acetate groups. It is not intended that the present invention be limited to the foregoing protecting groups, in fact, any compatible protecting group may be used.

The term “portion”, as used herein in reference to a protein refers to a fragment of that protein. Protein fragments or portions may range in size from four amino acid residues to the entire amino sequence minus one amino acid.

The term “chimera” or “hybrid”, as used herein, refers to any polypeptide comprising two or more peptide sequences that physically interact. The peptide sequences includes those obtained from the same or from different species or may be synthetic.

The term “fusion”, as used herein, refers to any chimeric polypeptide comprising a protein of interest joined to an exogenous protein fragment (i.e., a fusion partner). The purpose of a fusion partner includes, but is not limited to, provide a cell targeting moiety, enhancing solubility or providing a purification “affinity tag”. If desired, the fusion partner may be removed after or during purification.

The term “homolog” or “homologous”, as used herein, refers to any polypeptide having a high degree of identity to a reference polypeptide between their primary sequence structure, tertiary structure identity, the active site, or the mechanism of action. For example, a homolog peptide or protein may have a greater than 60% sequence identity, more preferably greater than 75% sequence identity, and still more preferably greater than 90% sequence identity, with a reference polypeptide.

The term “substantial identity”, as used herein, refers to two peptide sequences, when optimally aligned (using, for example, programs such as GAP or BESTFIT) share at least 80% primary sequence structure identity, preferably at least 90% primary sequence structure identity, more preferably at least 95% primary sequence structure identity or more (e.g., for example, 99% sequence identity). Preferably, residue positions which are not identical are related by conservative amino acid substitutions.

The terms “variant” and “mutant”, as used herein when in reference to a polypeptide, refers to an amino acid sequence that differs from another amino acid sequence by one or more amino acids. The variant or mutant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant or mutant may have “non-conservative” changes (e.g., replacement of a glycine with a tryptophan). Other variants or mutants may also include amino acid deletions or insertions (i.e., additions), either singly or simultaneously. Guidance in determining which and how many amino acid residues may be substituted, inserted or deleted without abolishing biological activity may be found using computer programs well known in the art, for example, DNAStar software. Variants or mutants can be tested in functional assays. Variants or mutants may comprise less than a 10%, preferably less than a 5% and still more preferably less than a 2% difference in amino acid sequence.

The term “expression vector” or “expression cassette” as used herein, refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), a termination sequence, and a ribosome binding site, often along with other sequences. Eukaryotic cells are known to utilize promoters, enhancers, and termination and polyadenylation signals.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The terms also refer to the linkage of amino acid sequences in such a manner so that a functional protein is produced.

The term “transfection” as used herein refers to the introduction of foreign DNA into cells. Transfection may be accomplished by a variety of means including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

The term “transgenic animal” as used herein, refers to an animal that includes a transgene which is inserted into a cell and which becomes integrated into the genome either of somatic and/or germ line cells of the.

The term “transgene” means a DNA sequence which is partly or entirely heterologous (i.e., not present in nature) to the animal in which it is found, or which is homologous to an endogenous sequence (i.e., a sequence that is found in the animal in nature) and is inserted into the animal's genome at a location which differs from that of the naturally occurring sequence. Transgenic animals which include one or more transgenes are within the scope of this invention.

### BRIEF DESCRIPTION OF THE FIGURES

FIG. 1 presents several different embodiments for bioapplications of peptide-fluorochrome binding pairs.

FIG. 2 presents exemplary data showing a phage display selection and evolution of IQSPHFF phage. FIG. 2A. Fold enrichment of BSA-VT680-selected phage. FIG. 2B. Sequences of randomly selected phage clones isolated from biopanning rounds 2, 3, and 4. Note: Appearance of two families containing (Q)SP/TP and HXHH, and expansion of clone IQSPHFF.

FIG. 3 presents exemplary data showing molecular modeling of IQSPHFF binding to VT-680. FIG. 3A. Binding affinities of IQSPHFF phage to BSA-conjugated fluorochromes via ELISA. FIG. 3B. Schematic π-π bonding between the phenylalanines of the peptide and the indole or benzoindole subunit of VT680 FIG. 3C. Demonstrating how SPH amino acids might extend over the linker of VT680 to the opposite face of the fluorochrome. FIG. 3D. Illustrating a possible interaction of the peptide hydrophobic isoleucine and the hydrophobic alkene of the fluorochrome.

FIG. 4 illustrates molecular modeling of IQSPHFF peptide binding to representative fluorochromes. FIG. 4A. Suboptimal docking of VT680 with the IQSPHFF peptide (SEQ ID NO:1) (i.e., for example, the 3-carbon linker may be too short). FIG. 4B. Sub-optimal docking of VT680 with the IQSPHFF peptide (SEQ ID NO:1) (i.e., for example, possible isoleucine-hexanoic acid steric interference). FIG. 4C. Optimal docking of AF750 the IQSPHFF peptide (SEQ ID NO:1).

FIG. 5 presents exemplary data showing IQSPHFF high affinity binding to VT680. FIG. 5A. A representative sigmoidal dose response curve of IQSPHFF binding (K_{D}=0.53±0.12 SD nanomolar). Bars: ±SEM. FIG. 5B. Immunodetection of VT680 on glass slide via IQSPHFF phage and anti-M13 antibody. Integrated pixel density of TMB substrate deposition was generated by anti-M13-HRP reactions on spot dilutions of VT680.

FIG. 6 presents exemplary data demonstrating that IQSPHFF can target and image VT680 by fluorescence microscopy (20× objective, equal exposure, FITC channel) and FACS. FIG. 6A. Beads conjugated to VT680. FIG. 6B: Pixel topography rendition of FIG. 6A. FIG. 6C. Beads were conjugated with BSA; inset: Pixel topography rendition of FIG. 6C. FIG. 6D. A soluble IQSPHFF-Biotin peptide was incubated with VT-680-conjugated Bang's beads (red histogram) or BSA-conjugated Bang's beads (black histogram), then incubated with streptavidin-FITC and analyzed via flow cytometry.

FIG. 7 presents exemplary data demonstrating IQSPHFF phage detected and co-localized with a VT-680-conjugated imaging agent in tumor specimens. Representative fluorescent microscopy images (20× objective; captured from FITC labeled phage left, and Cy5.5 channels, center; merge, right) from ex vivo LLC xenograft tumors containing SPARC targeted phage bionanoparticles that were conjugated with VT680. FIG. 7A. IQSPHFF phage. FIG. 7B. negative control (i.e., wild type phage).

FIG. 8 presents exemplary data demonstrating that peptide-NIRF pairs allow the in vitro and in vivo detection of cells. FIG. 8A. HT1080 cells were incubated with vehicle alone or Carboxy NHS functionalized IQSPHFF peptide. Cells were then incubated with the —NHS derivatived benzindol fluorochrome VT680 (also referred to herein as GH680 when coupled with a Genhance® dye) or with pre-incubated Genhance®+unconjugated peptide. The presence of NIRF was detected via fluorescence plate reader. FIG. 8B. Upper Left: Freeze fracture scanning electron micrograph of HT1080 cells imaged in the accompanying fluorescence micrographs. Lower Left: Fluorescence micrograph of HT1080 cells expressing an IQSPHFF-Platelet Derived Growth Factor Receptor (PDGFR) fusion protein incubated with VT680; Inset: Showing close-up of cell membrane binding. Upper Right: Fluorescence micrography of HT1080 cells expressing an PDGFR-red fluorescent protein (dsRed) fusion protein. Lower Right: Co-localization of VT680 and dsRed fluorescence using a merged image of lower left and upper right images. FIG. 8C. Mice implanted with HT1080 cells expressing IQSPHFF peptide fused to PDGFR (right flank) or without expression (left flank) were injected with NIRF and then imaged via FMT.

FIG. 9 presents exemplary data demonstrating the analysis of IQSPHFF (SEQ ID NO: 1) peptide binding to NIRF via HPLC. Free NIRF (upper trace) or NIRF incubated with IQSPHFF peptide (lower trace) was analyzed via HPLC with a reverse phase C₁₈ column. Note the shift in retention time for NIRF incubated with peptide indicating the formation of a stable complex.

FIG. 10 presents several embodiments of phage labeling. FIG. 10A: Fluorochromes used for phage labeling. Listed are the mean numbers of fluorochromes from a single comparative experiment. The relative fluorescence signal at a 6-mm tissue depth of different fluorochrome-labeled phages was calculated from known spectral tissue absorption. Note the much higher fluorescence of 680- to 750-nm fluorochrome-labeled phages in tissues. FIG. 10B: Emission spectra of fluorescently labeled phages (excitation and color of data points as listed in FIG. 10A).

FIG. 11 presents exemplary data showing the characterization of IQ-tag peptide/GH680 interactions using BiaCore experiments. FIG. 11A: IQ-tag-biotin was immobilized on a streptavidin chip then decreasing concentrations of GH680 were flowed over the surface. Top trace: 640 nM; Bottom trace: 0 nM (i.e, buffer only); 2^{nd} to bottom trace: 40 nM. FIGS. 11B & C. Photochemical properties of GH680 (green trace) and GH680+IQ-tag (red trace). GH-680 binding to IQ-tag blue shifts the absorption by 14 nm.

FIG. 12 presents exemplary data showing that peptide-NIR dye binding pairs allows for the in vitro and in vivo detection of cells. FIG. 12A. Left Image: HEK-293T cells with (red area) or without (blue areas) the expression of dsRed and IQ-tag peptide fused to PDGFR. Images were taken via fluorescence microscopy using GH680. Right Image: Confocal microscopy showing membrane localization of GH680. Scale bar: 10 mm. FIG. 12B. Non-IQ tagged animals were injected tail vein with GH680 and organs removed at indicated time points post injection to quantify fluorochrome concentrations. FIG. 12C: Mice implanted with HEK-293T cells expressing IQ-tag peptide fused to PDGFR (right flank; red area) or without expression (left flank; blue area) were injected with GH680 then imaged via FMT.

### DETAILED DESCRIPTION

The present invention is related to compositions and methods to identify and use unique peptide sequences that interact with small molecule imaging agents. The invention utilizes alternative phage libraries conjugated to imaging agents for screening cyclic and linear peptide sequences that bind with high affinity. For example, the peptide sequences comprise submicromolar affinities for the imaging agents. In one embodiment, the present invention contemplates a composition comprising a high affinity peptide/imaging agent binding pair that will be useful in the diagnosis and treatment of medical conditions.

Recent advances in molecular and cell biology techniques directed to a number of human disorders have suggested novel therapeutics and changed clinical approaches at the patient level. Furtherance of the present understanding diseases and developing new therapies may be advanced by noninvasive high-resolution in vivo imaging technologies. For example, the in vivo monitoring of specific molecular and cellular processes (i.e., for example, gene expression, multiple simultaneous molecular events, progression or regression of cancer, and drug and gene therapy) can be achieved by optical imaging. Shah et al., “Molecular Optical Imaging: Applications Leading to the Development of Present Day Therapeutics” NeuroRx. 2(2):215-225 (2005).

In recent years, many advances have been made in high-resolution in vivo imaging methods, including: radionuclide imaging, such as positron emission tomography (PET) and single-photon emission tomography (SPECT), magnetic resonance (MR) imaging, and spectroscopy. Optical imaging techniques have used different physical parameters of light interaction with tissue and a number of different optical imaging approaches have been described. These techniques rely on fluorescence, absorption, reflectance, and/or bioluminescence as a source of contrast.

In one embodiment, the present invention contemplates using optical imaging techniques including, but not limited to, near-infrared fluorescence (NIRF) reflectance imaging and bioluminescence imaging.

### I. Visualization of Site-Specific Proteins

Optically based biomedical imaging techniques may include, but are not limited to, laser technology, sophisticated reconstruction algorithms, and imaging software originally developed for non-optical, tomographic imaging modes such as CT and MRI. Visible wavelengths may be used for optical imaging of surface structures by means of endoscopy and/or microscopy.

Near infrared wavelengths (i.e., for example, approximately 600-1000 nm) have been used in optical imaging of internal tissues, because near infrared radiation exhibits tissue penetration of up to about fifteen centimeters. Wyatt, “Cerebral oxygenation and haemodynamics in the fetus and newborn infant,” Phil. Trans. R. Soc. London B 352:701-706 (1997); and Tromberg et al., “Non-invasive measurements of breast tissue optical properties using frequency-domain photo migration” Phil. Trans. R. Soc. London B 352:661-667 (1997).

Advantages of near infrared imaging over other currently used clinical imaging techniques include, but are not limited to, the following: potential for simultaneous use of multiple, distinguishable probes (important in molecular imaging); high temporal resolution (important in functional imaging); high spatial resolution (important in in vivo microscopy); and safety (no ionizing radiation).

In near infrared fluorescence imaging, filtered light or a laser with a defined bandwidth can be used as a source of excitation light. The light may be continuous in intensity, pulsed, or may be modulated (for example by frequency or amplitude). The excitation light travels through body tissues but may remain near the surface, for example at the skin or at an endothelial surface. When the excitation light encounters a near infrared fluorescent molecule (“contrast agent”), the light is absorbed. The fluorescent molecule then emits light that has detectably different properties (i.e., spectral properties of the probe (slightly longer wavelength), e.g., fluorescence) from the excitation light. Despite good penetration of biological tissues by light, conventional near infrared fluorescence probes are subject to many of the same limitations encountered with other contrast agents, including low target/background ratios.

A number of reporter and imaging technologies have recently been developed to visualize site-specific proteins and cellular trafficking. Specific examples include tags such as: i) tetracysteine or hexahistidine motifs to bi-arsenicals (Adams et al., J Am Chem Soc, 124:6063-6076 (2002); ii) his-tags for Ni-NTA-conjugated fluorochromes (Guignet et al., Nat Biotechnol, 22:440-444 (2004); or iii) various forms of biotin ligases that are revealed with labeled avidins. Chen et al., Nat Methods, 2:99-104 (2005); and Tannous et al., Nat Methods, 3:391-396 (2006). Although it is not necessary to understand the mechanism of an invention, it is believed that it should be feasible to develop affinity ligands for commercially available NIR fluorochromes by using phage screening to identify novel, short peptide sequences.

### II. Chromophore Binding Peptides

In one embodiment, the present invention contemplates novel, high affinity sequences, with specificity for fluorochromes and which do not occur in nature. In one embodiment, the sequences are short, linear, and have subnano-subpicomolar affinity. The affinity of these peptides are much higher than the micromolar affinities for most antibodies directed against visible light fluorochromes (i.e., for example, FITC, RITC). In one embodiment, the peptides cross-react with a plurality of different far red and near infrared fluorochromes comprising different pharmacological profiles.

A variety of short peptide sequences having specificity for specific fluorochromes have been reported. Some of these peptide sequences are either poly-histidine sequences or short amino acid sequences with flanking cysteines and are limited to a micromolar affinity for a fluorochrome. For the most part, these particular fluorochrome-binding peptides are co-located in tandem and respectively bind to different fluorochromes such that fluorescence resonance energy transmission (FRET) measurements can be obtained. Ebright R., “Ultra-High Specificity Fluorescent Labeling” United States Patent Application Publication No. 2006/014153 (herein incorporated by reference).

Tandem fluorochrome imaging probes conjugated to “attachment moieties” have been described wherein the attachment moieties are defined as polypeptides including, but not limited to polylysine, albumins and/or antibodies. No specific sequences for attachment moieties, however, are provided nor is there any disclosure for high affinity binding moieties (i.e., for example, submicromolar affinities). Weissleder, R., “Activatable Imaging Probes” United States Patent Application Publication No. 2005/0169843 (herein incorporated by reference).

Quenched fluorochrome imaging probes that are conjugated to “targeting moieties” for specific tissue attachments have also been described. In general, quenched fluorochromes are conjugated to a peptide backbone having enzyme-sensitive sequences. In the presence of disease-associated enzymes (i.e., for example, prostate associated antigen; PSA), the backbone is cleaved thereby unquenching the fluorochromes. Targeting moieties are identified as monoclonal antibodies or receptor-binding polypeptides. Weissleder et al., “Intramolecularly-Quenched Near Infrared Fluorescent Probes” U.S. Pat. No. 6,592,847 (herein incorporated by reference).

Fluorochromes conjugated to moieties that bind specifically to apoptotic cells have been reported. These binding moieties are described as annexin, synaptotagmin, and membrane phospholipid antibodies. The reference does not disclose a high affinity small peptide having submicromolar (i.e., for example, nanomolar, picomolar etc.) affinity for a fluorochrome. Bogdanov et al., “In Vivo Imaging Of Apoptosis” United States Patent Application Publication No: 2004/0022731 (herein incorporated by reference).

In one embodiment, the present invention contemplates a composition comprising a plurality of transformed cells tagged by expression of high affinity small peptide sequences. In another embodiment, the high affinity small peptide sequences may be directly conjugated to the outer cell membrane of a cell. In yet another embodiment, a first fluorochrome was used to tag the cells by standard antibody targeting followed by administration of a high affinity small molecule peptide-second fluorochrome conjugate.

A. The IQSPHFF Sequence (SEQ ID NO:1)

In one embodiment, the present invention contemplates a consensus sequence comprising the amino acid sequence IQSPHFF (SEQ ID NO:1). In one embodiment, the consensus sequence is hydrophobic comprising at least three (3) hydrophobic residues and an ionizable residue. In one embodiment, the consensus sequence comprises a C- and/or an N-terminal extension. In one embodiment, the C- and/or N-terminal extension comprise at least one glycine. Although it is not necessary to understand the mechanism of an invention, it is believed that glycine may increase hydrophilicity and acts as a spacer between target proteins.

In one embodiment, a peptide comprising IQSPHFF (SEQ ID NO: 1) does not occur in the mammalian genome, thereby making its expression unlikely to be confounded with other proteins. For example, a BLAST search of the human and mouse proteomes matched 5 out of 7 amino acids to titin sequence (a giant polypeptide found in striated muscle cells). Molecular modeling with the titin peptide on a NIRF failed to demonstrate the same binding mode(s) as the IQSPHFF (SEQ ID NO:1) peptide. Therefore, it is unlikely that these five (5) matching amino acids (with a gap in the sequence similarity) would exhibit affinity for benzindol fluorochromes as does SEQ ID NO:1. For example, preliminary experiments using in vivo imaging of muscle has not shown any appreciable binding of the this titin 5-mer sequence to a benzindol fluorochrome (data not shown).

In one embodiment, the present invention contemplates a small peptide sequence (i.e., for example, IQSPHFF (SEQ ID NO: 1)) comprising a high affinity for benzindol fluorochromes (i.e., for example, an indocyanine fluorochrome). In one embodiment, the fluorochromes may include, but are not limited to, far red and near infrared fluorochromes (i.e., for example, Cy 2.5, Cy 5.5, VT680, and AF750). Although it is not necessary to understand the mechanism of an invention, it is believed that the binding affinity of IQSPHFF (SEQ ID NO:1) for benzindol fluorochromes is significantly higher (i.e., for example, in the nanomolar and/or picomolar range) than traditional antibody binding affinities (i.e., for example, in the micromolar range). It is further believed that cell tagging with such high affinity small peptide sequences provide tremendous advantages for targeted delivery of fluorochromes that dramatically increases the sensitivity for detecting diseased tissues (i.e., for example, tumor cells).

Although it is not necessary to understand the mechanism of an invention, it is believed that the identified IQ-tag peptide (IQSPHFF: SEQ ID NO: 1) is unique and has a sub-nanomolar binding affinity for the NIR fluorochrome GH680, due to a number of optimal binding interactions between the peptide and benzindolium dye. It is further believed that, due to alanine scanning results, that the I, S, and F positions may play constitutive roles in the binding ability of an IQ-tag with a target. According to results presented herein obtained from molecular modeling studies of the docked ligand, π-stacking between the phenylalanine residues of IQ-tag and the (benz)indolium subunit of the fluorophore yields preferential orientation of the peptide on the dye. The next three amino acids, SPH, extend over the alkene linker of the dye to the opposite face with the hydrophilic portions directed away from the core. Finally, the hydrophobic N-terminal isoleucine is able to interact with the similarly hydrophobic polymethine linker. When the binding of the peptide to various other cyanine dyes (AF750, Cy5.5, Cy3.5) is investigated, varied affinity constants are obtained. These differences in affinity, as well as alterations to the peptide sequence, could be used to identify IQ-tag like sequences with even higher affinity or subsets with affinity for specific NIR fluorochromes. For example, the use of unnatural amino acids or additional screens with mRNA display could be used to yield additionally evolved compounds. Wilson et al., “The use of mRNA display to select high-affinity protein-binding peptides” Proc Natl Acad Sci USA 98: 3750-3755 (2001). The somewhat hydrophobic IQ-tag can also be modified at either its N- or C-terminus in order to modulate the polarity of the peptide.

In one embodiment the present invention contemplates an amino acid sequence comprising I-X₁-S-P-X₂-X₃-X₄ (SEQ ID NO: 17), wherein X₁ is either Q, T, or P; X₂ is either H, P, S, or N, X₃ is either F, L, I or P; and X₄ is either F, L, I, or T.

In one embodiment, the present invention contemplates an amino acid sequence comprising I-X₁-T-P-X₂-X₃-X₄ (SEQ ID NO: 18), wherein X₁ is either Q, T, or P; X₂ is either H, P, S, or N, X₃ is either F, L, I, or P; and X₄ is either F, L, I, or T.

B. The HHSHRHH Sequence (SEQ ID NO: 16)

In one embodiment, the present invention contemplates a consensus sequence comprising the amino acid sequence HHSHRHH (SEQ ID NO:16). In one embodiment, the consensus sequence is hydrophilic comprising at least three (3) hydrophilic residues and an ionizable residue. In one embodiment, the consensus sequence comprises a C- and/or an N-terminal extension. In one embodiment, the C- and/or N-terminal extension comprise at least one glycine. Although it is not necessary to understand the mechanism of an invention, it is believed that glycine may increase hydrophilicity and acts as a spacer between target proteins.

In one embodiment, a peptide comprising HHSHRHH (SEQ ID NO:16) does not occur in the mammalian genome, thereby making its expression unlikely to be confounded with other proteins. For example, a BLAST search of the human and mouse proteomes found no significant similarities. However, two exact matches were found in two plant proteins; Populus (xyloglycan endotransglycosylase percursor) and Medicago (unknown function); and one exact match in a Drosophila protein (CG15489-PA) and a Synechoccous protein (hypothetical protein WH7805_{—}01442).

In one embodiment, the present invention contemplates a small peptide sequence (i.e., for example, HHSHRHH (SEQ ID NO:16)) comprising a high affinity for benzindol fluorochromes (i.e., for example, an indocyanine fluorochrome). In one embodiment, the fluorochromes may include, but are not limited to, far red and near infrared fluorochromes (i.e., for example, Cy 2.5, Cy 5.5, VT680, and AF750). Although it is not necessary to understand the mechanism of an invention, it is believed that the binding affinity of HHSHRHH (SEQ ID NO:16) for benzindol fluorochromes is significantly higher (i.e., for example, in the nanomolar and/or picomolar range) than traditional antibody binding affinities (i.e., for example, in the micromolar range). It is further believed that cell tagging with such high affinity small peptide sequences provide tremendous advantages for targeted delivery of fluorochromes that dramatically increases the sensitivity for detecting diseased tissues (i.e., for example, tumor cells).

In one embodiment, the present invention contemplates an amino acid sequence comprising H-X₁-X₂-H-X₃-X₄-X₅ (SEQ ID NO: 19), wherein X₁ is either H or G; X₂ is either S or H; X₃ is either R, L or P; and X₄ is either H, S, or R; and X₅ is either H, M, or Y.

C. Alternative Sequences

In one embodiment, the present invention contemplates a peptide capable of high affinity binding to a fluorochrome comprising between approximately 5-15 amino acids. In another embodiment, the present invention contemplates a peptide capable of high affinity binding to a fluorochrome comprising between approximately 7-20 amino acids.

In one embodiment, the present invention contemplates a peptide capable of high affinity binding to a fluorochrome comprising X₁-X₂-X₃-X₄-X₅-X₆-X₇-X₈-X₉-X₁₀-X₁₁, wherein: wherein X₁ is either S, T, or nothing; wherein X₂ is either T, N, L, V, E, or nothing; X₃ is either H, K, T, L, M, N, G, R, P, F, or nothing; X₄ is either Q, T, H, P, S, G, A, I, D, N, E, or nothing; X₅ is either S, T, H, L, I, E, Y, N, M, R, D, G, A, or W; X₆ is either P, T, H, S, K, F, or E; and X₇ is either L, P, S, T, N, G, F, D, Y, M, or C; X₈ is either P, S, G, E, Q, A, T, K, V, D, Y, M, I, R, F, L, or nothing; X₉ is either F, R, P, M, T, Y, K, I, D, S, H, V, A, or nothing; X₁₀ is either P, T, Y, W, or nothing; and X₁₁ is S or nothing.

In one embodiment, the present invention contemplates a peptide capable of high affinity binding to a fluorochrome comprising X₁-X₂-X₃-X₄-X₅-X₆-X₇-X₈-X₉-X₁₀, wherein: wherein X₁ is either K, M, S, or nothing; wherein X₂ is either I, H, A, F, S, W, T, R, M, D, G, or nothing; X₃ is either Q, H, G, K, E, T, L, S, R, G, A, W, or nothing; X₄ is either S, H, L, V, I, P, Q, or E; X₅ is either P, H, G, S, R, or Q; X₆ is either H, L, T, P, Y, R, E, N, M, D, F, N, I, or A; and X₇ is either F, H, S, Q, R, P, T, Y, A, N, or V; X₈ is either F, M, P, Y, R, S, L, T, H, V, or nothing; X₉ is either H, V, I, L, or nothing; and X₁₀ is either K or nothing.

In one embodiment, the present invention contemplates a peptide capable of high affinity binding to a fluorochrome comprising X₁-X₂-X₃-X₄-X₅-X₆-X₇-X₈, wherein: wherein X₁ is either I, H, V, K, G, or nothing; X₂ is either Q, H, N, G, or D; X₃ is either S, H, V, or P; X₄ is either P, H, S, or T; X₅ is either H, R, L, T, or V; X₆ is either F, H, P, S, or L; and X₇ is either F, H, M, T, L, E, or S; and X₈ is either P or nothing.

D. Peptides Comprising Cell Targeting Moieties

In one embodiment, a peptide comprising a high affinity binding site for a fluorochrome may further comprise a cell targeting moeity. In one embodiment, the cell targeting moiety is conjugated to the C-terminal end of the peptide. In one embodiment, the cell targeting moiety comprises an antibody. In one embodiment, the cell targeting moiety comprises a cell receptor. In one embodiment, the cell receptor comprises a platelet derived growth factor receptor. In one embodiment, the cell targeting moiety comprises a functional group, wherein the functional group attaches to the cell. In one embodiment, the functional group comprises a carboxylic acid. In one embodiment, the functional group comprises an amide. In one embodiment, the functional group comprises a phosphate group. In one embodiment, the functional group comprises a sulfhydryl group. In one embodiment, the functional group comprises an acetyl group.

Antibodies capable of binding to the peptides of the present invention may be obtained commercially (see, Examples) or generated by immunization (i.e., for example, polyclonal antibodies) or generated by hybridoma expression platforms (i.e., for example, monoclonal antibodies).

In addition, chimeric antibodies may be produced, for example, by splicing mouse antibody genes to human antibody genes. Single chain antibodies are also contemplated as well as antibody fragments generated by pepsin digestion of the antibody molecule or by reduction of the disulfide bridges of the Fab fragments. For the production of antibodies, any antigenic portion of an amino acid sequence contemplated herein (i.e., for example, SEQ ID NOs:1 or 16) can be used either alone, or fused with amino acids of another protein (i.e., for example, glutathione to produce an antibody against a chimeric molecule). Such antibodies include, but are not limited to, polyclonal, monoclonal, chimeric, single chain, Fab fragments and fragments produced by a Fab expression library. Neutralizing antibodies, i.e., those which inhibit dimer formation, are especially preferred for diagnostics and therapeutics.

Antigenic polypeptides may be used for antibody generation and need not retain biological activity. Antigenic peptides used to generate specific antibodies may have an amino acid sequence comprising at least five amino acids, preferably at least 10 amino acids. Preferably, they should mimic at least a portion of the amino acid sequence of the natural protein and may contain the entire amino acid sequence of a small, naturally occurring molecule. Short stretches of an amino acid sequence may be fused with those of another protein including, but not limited to, keyhole limpet hemocyanin and antibody produced against a chimeric molecule.

For the production of antibodies, various hosts including goats, rabbits, rats, mice, etc may be immunized by injection with a full-length protein or any portion, fragment or oligopeptide which retains immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guefin) and Cornebacterium parrum are potentially useful adjuvants.

Monoclonal antibodies may be prepared using any technique which provides for the production of antibody molecules by continuous cell lines in culture. These include, but are not limited to hybridoma techniques. Koehler et al. Nature 256:495-497 (1975); Kosbor et al., Immunol Today 4:72 (1983); Cote et al., Proc Natl Acad Sci 80:2026.-2030 (1983); Cole et al., In: Monoclonal Antibodies and Cancer Therapy, Alan R Liss Inc, New York, N.Y., pp 77-96 (1985) (all references incorporated herein by reference).

E. Degradation Resistant Peptides

In one embodiment, it is contemplated that the peptides of the present invention are made more resistant to degradation by protease. This can be achieved several ways. In one embodiment, the proteins of the present invention may be made cyclic by attaching cytosine residues to the amine and carboxyl termini or by attaching crosslinkers. In another embodiment, the peptide bond between the amino acids can be replaced with a CH₂ group, or the like. In another embodiment, commercially available protecting groups can be attached to the amino and carboxyl termini. In yet another example, one or more L-amino acids can be replaced with D-amino acids.

In another embodiment, it is contemplated that the peptides of the present invention comprise modified amino acids having at least one non-amino acid moieties (i.e., for example, forming a peptoid). In one embodiment, modified amino acids comprise side chains that are different from the non-modified amino acid. Glycine, for example, can be replaced with an N-alkylated glycine such as N-isobutylglycine. Kwak, et al., “Triple Helical Stabilities Of Guest-Host Collagen Mimetic Structures,” Bioorg Med Chem. 7:153-60 (1999). Alternatively, one or more amino acids maybe completely replaced with one or more non-amino acid moieties. Pueyo, et al., “A Mimetic Of The RGDF-Peptide (arginine-glycine-aspartic acid-phenylalanine) Blocks Aggregation And Flow-Induced Platelet Deposition On Severely Injured Stenotic Arterial Wall. Effects On Different Animal Models And In Humans” Thromb Res. 81:101-112 (1996).

F. Protein Mimetics

In one embodiment, the present invention contemplates a protein mimetic compound having a high affinity binding site for a fluorochrome. A protein mimetic may comprise a compound mimicking the necessary conformation for recognition and docking of a high affinity binding sites of the present invention are contemplated as within the scope of this invention. For example, mimetics of an IQSPHFF peptide is contemplated.

A variety of designs for such mimetics are possible. For example, cyclic IQSPHFF-containing peptides, in which the necessary conformation for binding is stabilized by nonpeptides, are specifically contemplated. U.S. Pat. No. 5,192,746 to Lobl, et al, U.S. Pat. No. 5,169,862 to Burke, Jr., et al, U.S. Pat. No. 5,539,085 to Bischoff, et al, U.S. Pat. No. 5,576,423 to Aversa, et al, U.S. Pat. No. 5,051,448 to Shashoua, and U.S. Pat. No. 5,559,103 to Gaeta, et al, all hereby incorporated by reference, describe multiple methods for creating such compounds.

Synthesis of nonpeptide compounds that mimic peptide sequences is also known in the art. Eldred, et al, (J. Med. Chem. 37:3882 (1994)) describe nonpeptide antagonists that mimic the Arg-Gly-Asp sequence. Likewise, Ku, et al, (J. Med. Chem. 38:9 (1995)) give further elucidation of the synthesis of a series of such compounds. Such nonpeptide compounds that mimic IQSPHFF peptides are specifically contemplated by the present invention.

The present invention also contemplates synthetic mimicking compounds that are multimeric compounds that repeat the relevant peptide sequence. In one embodiment of the present invention, it is contemplated that the relevant peptide sequence is S-P-H-F; in another embodiment, the relevant peptide sequence is I-Q-S-P. Peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea.

It can be necessary to protect potentially reactive groups other than the amino and carboxyl groups intended to react. For example, the α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to the above-described IQSPHFF-containing peptides. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant. W. R. Pearson and D. J. Lipman, Proc. Natl. Acad. Sci. (USA), 85:2444-2448 (1988); D. J. Lipman and W. R. Pearson, Science, 227:1435-1441 (1985). In the present invention, synthetic polypeptides useful in disease therapy are those peptides with statistically significant sequence homology and similarity (Z value of Lipman and Pearson algorithm in Monte Carlo analysis exceeding 6).

### III. Chromophores

Chromophores useful in the present invention include, but are not limited to, near infrared chromophores such as Cy5.5, Cy5, Cy7, IRD41, IRD700, NIR-1, IC5-OSu, LaJolla Blue, Alexaflour 660, Alexflour 680, FAR-Blue, FAR-Green One, FAR-Green Two, ADS 790-NS, ADS 821-NS, indocyanine green (ICG) and analogs thereof, indotricarbocyanine (ITC), chelated lanthanide compounds that display near infrared optical properties, and fluorescent quantum dots (zinc sulfide-capped cadmium selenide nanocrystals) (e.g., QuantumDot Corporation; www.qdots.com). The chromophores can be covalently linked to spacers, using any suitable reactive group on the chromophore and a compatible functional group on the spacer. A chromophore according to the present invention may comprise a targeting moiety including, but not limited to, an antibody, antigen-binding antibody fragment, a receptor-binding polypeptide, a receptor-binding polysaccharide, or a hydrophobic region.

Organic fluorochromes are small probes and have used in immunofluorescence, analytical testing, and emerging imaging technology. In particular, far red and near infrared fluorochromes (NIRFs) incorporating benzindol rings are useful for in vivo imaging applications as light penetrates tissue more efficiently in this range and because tissue autofluorescence is much lower. Ntziachristos et al., Nat Biotechnol, 23:313-320 (2005); and Weissleder et al., Nat Med, 9:123-128 (2003). For example, NIRFs have been used in vivo for: i) detection of early forms of atherosclerosis. Chen et al., Circulation, 111:1800-1805 (2005); ii) detection of cancer (Weissleder et al., Nat Biotechnol, 17:375-378 (1999); and iii) tracking of specific cell types including, but not limited to, macrophages (Weissleder et al., Nat Biotechnol, 23:1418-1423 (2005); endothelial cells (Kelly et al., Circ Res, 96:327-336 (2005); and smooth muscle, fibroblasts, or stem cells (Kim et al., Stroke, 35:952-957 (2004).

Although it is not necessary to understand the mechanism of an invention, it is believed that organic fluorochromes, as contemplated herein, do not suffer from many of the detrimental aspects of FP. Fluorochromes have been used in immunofluorescence, analytical testing, and other emerging imaging applications. In particular, far red (FR) and near infrared (NIR) fluorochromes containing (benz)indolium subunits have proven useful in a multitude of in vivo imaging applications, as light penetrates tissue more efficiently and because tissue autofluorescence is much lower in this range. Ntziachristos et al., “Looking and listening to light: the evolution of whole-body photonic imaging.” Nat Biotechnol 23:313-320 (2005); and Weissleder et al., “Shedding light onto live molecular targets” Nat Med 9:123-128 (2003). To expand the utility of these fluorochromes, it would be useful to develop biological ligands tailored specifically to the structure of the dye, ideally with affinity constants surpassing those of monoclonal antibodies. In one embodiment, the present invention contemplates short peptides specific to organic fluorochromes identified through library screening that may: a) provide a minimally invasive way of “tagging” proteins, b) enable imaging of intracellular proteins, and c) provide specificity since endogenous proteins should not recognize NIR dyes.

In one embodiment, the fluorochrome comprises a polymeric backbone and a plurality of near infrared fluorochromes covalently linked to the backbone. In one embodiment, the backbone can be any biocompatible polymer, including, but not limited to, a polypeptide, a polysaccharide, a nucleic acid, or a synthetic polymer. For embodiments where polypeptides are useful as a backbone, these polypeptides may include, but are not limited to, polylysine, albumins, and antibodies. For embodiments where synthetic polymers are useful as a backbone, these synthetic polymers include, but are not limited to, polyglycolic acid, polylactic acid, poly(glycolic-colactic) acid, polydioxanone, polyvalerolactone, poly-.epsilon.-caprolactone, poly(3-hydroxybutyrate, poly(3-hydroxyvalerate) polytartronic acid, and poly(β-malonic acid).

Probe backbone design will depend on considerations such as biocompatibility (e.g., toxicity and immunogenicity), serum half-life, useful functional groups (for conjugating fluorochromes, spacers, and protective groups), and cost. Useful types of backbone include polypeptides (polyamino acids), polyethyleneamines, polysaccharides, aminated polysaccharides, aminated oligosaccharides, polyamidoamines, polyacrylic acids and polyalcohols. In some embodiments, the backbone comprises a polypeptide formed from L-amino acids, D-amino acids, or a combination thereof. Such a polypeptide can be, e.g., a polypeptide identical or similar to a naturally occurring protein such as albumin, a homopolymer such as polylysine, or a copolymer such as a D-tyr-D-lys copolymer. When lysine residues are present in the backbone, the ε-amino groups on the side chains of the lysine residues can serve as convenient reactive groups for covalent linkage of fluorochromes and spacers. When the backbone is a polypeptide, preferably the molecular weight of the probe is from 2 kD to 1000 kD. More preferably, its molecular weight is from 4 kd to 500 kd.

A backbone may be chosen or designed so as to have a suitably long inherent in vivo persistence (i.e., for example, half-life). Therefore, protective chains are not necessary in some embodiments of the invention. Alternatively, a rapidly-biodegradable backbone such as polylysine can be used in combination with covalently-linked protective chains. Examples of useful protective chains include polyethylene glycol (PEG), methoxypolyethylene glycol (MPEG), methoxypolypropylene glycol, polyethylene glycol-diacid, polyethylene glycol monoamine, MPEG monoamine, MPEG hydrazide, and MPEG imidazole. The protective chain can also be a block-copolymer of PEG and a different polymer such as a polypeptide, polysaccharide, polyamidoamine, polyethyleneamine or polynucleotide. In one embodiment, the backbone comprises a synthetic, biocompatible polymer. Holland et al., “Biodegradable Polymers” Advances in Pharmaceutical Sciences 6:101-164 (1992). In one embodiment, a backbone-protective chain combination is methoxypoly(ethylene)glycol-succinyl-N-E-poly-L-lysyine (PL-MPEG). Bogdanov et al., U.S. Pat. No. 5,593,658; and Bogdanov et al., Advanced Drug Delivery Reviews 16:335-348 (1995) (both herein incorporated by reference).

Expansion of the utility of these fluorochromes to develop novel applications requires developing biological affinity ligands having affinity constants surpassing those of monoclonal antibodies. The development of peptide sequences specific to organic fluorochromes through screening evolutionary libraries may be a way to circumvent previous issues with visualization of proteins by: i) providing a much less invasive way of “tagging” a protein of interest; ii) allowing the visualization of intracellular proteins with few manipulations; iii) providing specificity since endogenous proteins should not recognize the organic fluorochromes; and iv) the selection process allows for the generation of peptide sequences with high affinity.

In one embodiment, the present invention contemplates a method comprising phage screening to identify high affinity small peptide sequences for fluorochrome probes. In one embodiment, the peptide comprises a high affinity for benzindol fluorochromes. In one embodiment, the phage screening creates a small 7-mer library that converges onto a single peptide sequence comprising subnanomolar affinity for benzindol fluorochromes.

In one embodiment, the present invention contemplates a method comprising using a small peptide having high affinity for a fluorochrome probe for qualitative techniques including, but not limited to, immunostaining, analytical testing or cell tracking. In one embodiment, the fluorochrome probe further comprises membrane translocation signals and/or other ligands that facilitate internalization processes.

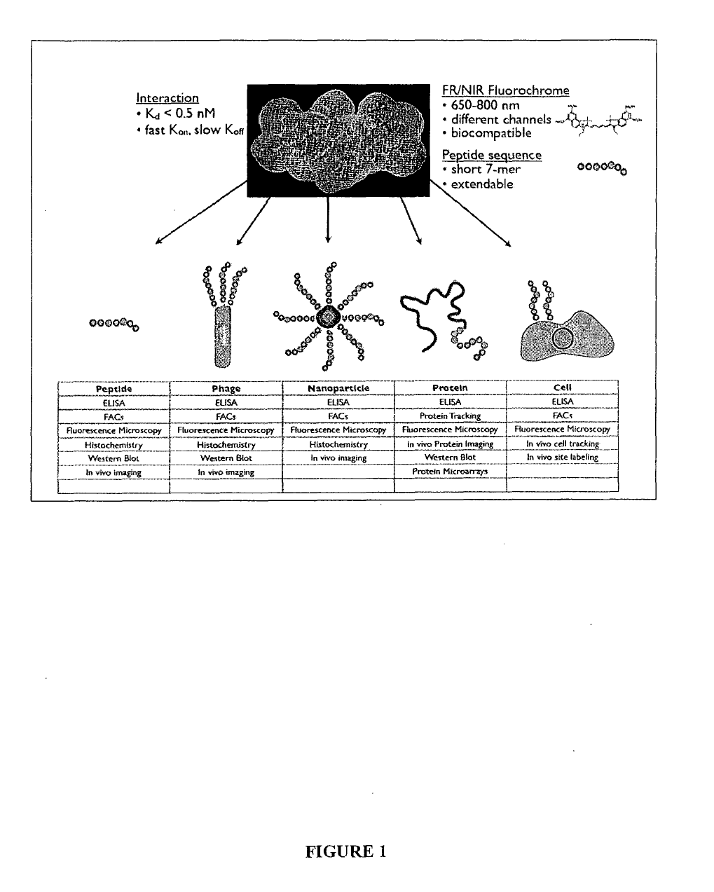

In one embodiment, the present invention contemplates a method comprising using a small peptide having high affinity for a fluorochrome probe for analytical techniques including, but not limited to, FACS, ELISA, histochemistry, and in vitro and in vivo protein and cell labeling. See, FIG. 1. In one embodiment, the fluorochrome probe further comprises membrane translocation signals and/or other ligands that facilitate internalization processes. In one embodiment, the small peptides comprise a submicromolar high affinity for the fluorochrome probes.

In one embodiment, the cellular delivery of fluorochromes may be enhanced by the addition of membrane translocation signals, or other ligands that facilitate internalization processes.

In one embodiment, the present invention contemplates an imaging fluorochrome comprising excitation and emission wavelengths in the near infrared spectrum are preferred, i.e., 650-1300 nm. Although it is not necessary to understand the mechanism of an invention, it is believed that use of this portion of the electromagnetic spectrum maximizes tissue penetration and minimizes absorption by physiologically abundant absorbers such as hemoglobin (<650 nm) and water (>1200 nm). It is further believed that ideal near infrared chromophores for in vivo use exhibit the following characteristics: (1) narrow spectral characteristics, (2) high sensitivity (quantum yield), (3) biocompatibility, and (4) decoupled absorption and excitation spectra. Table 1 summarizes information on the properties of some commercially available near infrared chromophores.

TABLE 1
Exemplary Near Infrared Chromophores

| | λ (nm) | λ (nm) | Mol. | Extinct. | Quantum |
|---|---|---|---|---|---|
| Fluorochrome | excitation | emission | Wt. | Coef. | yield % |
| Cy5.5 | 675 | 694 | 1128.41 | 250000 | 28.0 |
| Cy5 | 649 | 670 | 791.99 | 250000 | 28.0 |
| Cy7 | 743 | 767 | 818.02 | 200000 | 28.0 |
| IRD41 | 787 | 807 | 925.10 | 200000 | 16.5 |
| IRD700 | 685 | 705 | 704.92 | 170000 | 50.0 |
| IC5-OSu | 641 | 657 | 630.3 | NA | NA |
| NIR-1 | 663 | 685 | 567.08 | 75000 | NA |
| LaJolla Blue | 680 | 700 | 5000.00 | 170000 | 70.0 |
| Alexa Fluor 660 | 663 | 690 | 1100 | 132000 | NA |
| Alexa Fluor 680 | 679 | 702 | 1150 | 184000 | NA |
| ADS 790 NS | 791 | >791 | 824.07 | NA | NA |
| ADS 821 NS | 820 | >820 | 924.07 | NA | NA |
| Far-Blue | 660 | 678 | 825 | 150000 | NA |
| Far-Green One | 800 | 820 | 992 | 150000 | NA |
| Far-Green Two | 772 | 778 | | 150000 | NA |
| ICG | 780 | 812 | 774.98 | 115000 | 1.2 |
| ITC* | 753 | 790 | 1089 | 201000 | 6.6 |

*See, WO 98/47538

Examples of several commercially available fluorochrome sensors/indicators molecules are listed in Table 2.

TABLE 2
Fluorochromes Useful For Ion Detection

| | λ (nm) | λ (nm) | | Best Detection |
|---|---|---|---|---|
| Fluorochrome | excitation | emission | Analyte | Mode |
| DHPN | 360/420 | 455/512 | H⁺ | Emission Ratio |
| BCECF | 440/490 | 530 | H⁺ | Excitation Ratio |
| SNARF-1 | 517/576 | 587/640 | H⁺ | Emission Ratio |
| PBFI | 340/350 | 530 | K⁺ | Excitation Ratio or |
| | | | | Intensity |
| SBFI | 340/385 | 530 | Na⁺ | Excitation Ratio |
| Fluo-3 | 500 | 530 | Ca²⁺ | Intensity |
| Rhod-2 | 522 | 581 | Ca²⁺ | Intensity |
| OxyPhor-R2 | 419/524 | | O₂ | Lifetime |

Several of these fluorochrome molecules are commercially available as succimidyl esters that can be easily conjugated to primary amine groups, e.g., of peptides or other biologically compatible molecules. Although near-infrared fluorochromes are useful, it will be appreciated that the use of fluorochromes with excitation and emission wavelengths in other spectrums, such as the visible light spectrum, can also be employed in the compositions and methods of the present invention.

Many of these molecules, and others like them, have been used in vivo. For example. BCECF has been used in vivo to measure the pH of gastrointestinal mucosa, which is an important factor in the detection of hypoxia-induced dysfunctions (Marechal et al., Photochem. Photobiol., 1999, 70:813-819) as well as for intracellular pH measurement during cerebral ischemia and reperfusion (Itoh et al., Keio J. Med., 1998, 47:37-41) and for non-invasively monitoring the in vivo pH in conscious mice (Russell et al., Photochem. Photobiol., 1994, 59:309-313). In addition, 5,6-carboxyfluorescein has been used in vivo to measure the pH of tumor tissue (Mordon et al., Photochem. Photobiol., 1994, 60:274-279.) The phosphorescent oxygen probes Green 2W and Oxyphor R2 have been used to measure the oxygenation of cancerous tissue (Lo et al., Adv. Exp. Med. Biol., 1997, 411:577-583; Wilson et al., Adv. Exp. Med. Biol., 1998, 454:603-609), while the hydrogen peroxide probe 2′-7′-dichlorofluoroscein has been used in vivo to measure the level of oxidative stress (Watanabe, S., Keio J. Med., 1998, 47:92-98).

In another embodiment, probes can be activated by changes in H⁺ ion concentration or pH changes. Probes can be designed to contain spacers that are cleaved when physiological pH values are lowered. Examples of such spacers include, but are not limited to, alkylhydrazones, acylhydrazones, arylhydrazones, sulfonylhydrazones, imines, oximes, acetals, ketals, or orthoesters.

Analyte activation can be used to detect and/or evaluate many diseases or disease-associated conditions. The redistribution of analytes including, but not limited to, potassium, sodium, and calcium is often indicative of certain physiological processes and diseases including, but not limited to, hypoxia, ischemia, cerebrovascular ischemia due to stroke, embolism or thrombosis, ischemia of the colon, vascular ischemia due to coronary artery disease of heart disease, ischemia due to physical trauma, poisons, ischemia associated with encephalopathy; or renal ischemia. In addition, tumors are characterized by low pH values by comparison to normal tissue as well as inflammation, particularly inflammation caused by foreign pathogens.

### IV. Peptide-Fluorochrome Applications

The present invention contemplates a peptide-fluorochrome binding pair system that is quite adaptable to a number of different mono- and multivalent interactions. For example, peptide-fluorochrome binding pair system comprising cell-permeable fluorochromes may be useful for applications including, but not limited to, soluble peptide identification, prokaryotic surface display, nanomaterial and/or nanosensor multivalent display, site-specific protein expression, or eukaryotic cell tagging. In turn, each one of these examples have a plurality of practical variations.

As mentioned above, the “dye-sensing” ability of an IQ-tag system creates compatibilities with a number of different modalities. For example, cell permeable fluorochromes may be used for detection of: i) a soluble peptide fused to cytoplasmic proteins; ii) expression of a peptide on prokaryotic cells; iii) multivalent display of IQ-tag on a variety of nanomaterials and sensors; iv) site-specific protein expression; and v) eukaryotic cell tagging applications. Furthermore, an IQ-tag sequence, modified with a non-cyanine dye-based fluorochrome, radioisotope, or biotin, could also be used as an amplification strategy to determine (benz)indolium fluorochrome localization and/or concentration. Another possibility, as shown herein using cell experiments, provides inclusion of an IQ-tag sequence as a handle on an engineered protein to permit in vitro or in vivo protein or cellular imaging. For example, if a tagged protein comprises a target including, but not limited to, an internalizable receptor, growth factor, viral coat protein, or peptide toxin, an avenue for detailed and live imaging of intracellular receptor or drug trafficking becomes plausible. The novel IQ-tag technology described herein will be particularly useful for in vivo experimentation, as the transparency of animal tissue increases at far-red and near-infrared wavelengths. In total, IQ-tag answers a long felt need and represents a system, which can have immediate, broad biological application.

Consequently, the various embodiments of the present invention should find broad applicability for many biological applications. See, FIG. 1. Of these, a few examples are discussed below in more detail.

A. Fluorescence Activated Cell Sorting (FACS)

In mammals, the use of fluorescence-activated cell sorting (FACS) has driven much of the progress in subset discrimination and functional analysis for cellular analysis. Herzenberg et al., Clin. Chem. 48:1819-1827 (2002). Current three-laser, “multidimensional”, FACS machines enable up to 14 simultaneous single-cell measurements, namely 2 light scatters and 12 fluorescent surface/intracellular markers. Baumgarth et al., J. Immunol. Methods 243:77-97 (2000); De Rosa et al., Nat. Med. 7:245-248 (2001); and Perez et al., Nat. Biotechnol. 20:155-162 (2002). FACS also enables the sorting of subsets of interest and their further use in in vitro and in vivo assays. For example, FACS has been applied to freshly harvested Drosophila hemocytes, featuring a one-parameter analysis of hemolymph (blood surrogate) cells for surface antibody reactivity. Kurucz et al., Proc. Natl. Acad. Sci. USA 100:2622-2627 (2003). The ability to perform single-cell analyses and sorts on freshly harvested hemocytes and to use sorted hemocyte fractions with existing molecular tools for further in vitro or in vivo studies provides a useful experimental tool.

A generic FACS method has been described that enables the detection and multidimensional analysis of live cell subsets. Tirouvanziam et al., “Fluorescence-activated cell sorting (FACS) of Drosophila hemocytes reveals important functional similarities to mammalian leukocytes” Proc. Natl. Acad. Sci. USA 101; 2912-2917 (2004). Various FACS probes are listed in Table 3.

TABLE 3
Fluorescence-Activated Cell Sorting Probes

| Name | Source | Titer | Measurement | Spectra * | Laser |
|---|---|---|---|---|---|
| Annexin V | BD Pharmingen | 20.0 ng/ml | Surface phosphatidylserine (apoptosis) | variable ^{†} | variable ^{†} |
| Diaminofluorescein diacetate | Sigma | 1.0 μM | Intracellular nitric oxide and peroxynitrite | 495/515 ^{‡} | 488 nm |
| Dihydrorhodamine 123 | Sigma | 1.0 μM | Intracellular reactive oxygen species | 507/529 ^{‡} | 488 nm |
| C₁₂RG ^{§} | Molecular Probes | 22.5 μM | Intracellular LacZ reporter activity | 571/585 | 595 nm |
| Fluo3-FF-AM | Sigma | 0.3 μM | Intracellular calcium | 462/526 ^{‡} | 488 nm |
| GFP | | | Intracellular GFP reporter activity | 489/508 ^{‡} | 488 nm |
| Heat-killed bacteria | Molecular Probes | | Phagocytosis | variable ^{†} | variable ^{†} |
| Helix pomatia lectin | Sigma | 5.0 μM | Surface α-N-acetyl galactosamine | variable ^{†} | variable ^{†} |
| Monochlorobimane | Molecular Probes | 20.0 μM | Intracellular glutathione | 380/461 | 407 nm |
| Propidium iodide | Molecular Probes | 2.0 μg/ml | Cell-impermeable DNA probe (dead cells) | 495/637 | 488 nm |
| Soybean agglutinin | Molecular Probes | 2.0 μg/ml | Surface α/β-N-acetyl galactosamine | variable ^{†} | variable ^{†} |
| | | | and galactopyranoside | | |
| Wheat germ agglutinin | Molecular Probes | 2.0 μg/ml | Surface N-acetyl glucosamine and N-acetyl | variable ^{†} | variable ^{†} |
| | | | neuraminic acid | | |

* Peak excitation and emission in nm.
^{†} These probes are not inherently fluorescent and can be conjugated to various fluorochromes and, thus, excited and measured at chosen wavelengths.
^{‡} These probes are incompatible with each other, because of similar emission peaks.
^{§} C₁₂RG is part of the ImaGeneRed ® kit.

GFP and β-galactosidase (LacZ) reporters, however, are widely used, can be precisely quantified. Tickoo et al., Curr. Opin. Pharmacol. 2:555-560 (2002).

Staining conditions (i.e., for example, buffer, pH, temperature, and incubation time) may be optimized. For example, monochlorobimane (MCB) may be optimized by titration on larval hemocyte suspensions, mbn-2 cells, and S2 cells. The optimization conditions found for MCB are also useful for titration of other probes. Possible biochemical/optical interactions between probes are checked by comparing fluorescences measured with stains applied either alone or in combination with one another. Optimized conditions yield viable cells, generate reproducibility of measured signals while minimizing washing steps. Cells can be incubated with a probe for 20 min at room temperature and in the dark. Staining medium may comprise, for example, Schneider's medium, pH 6.5, with 2.5 mM probenecid (Sigma) to limit active probe efflux. Cells may be washed once with 10 ml of ice-cold staining medium and centrifuged for 5 min at 4° C. at 1,500 rpm (LX-130 centrifuge; Tomy, Tokyo). Supernatant is then discarded, and the pellet kept on ice in the dark. Antibody-staining may be performed subsequently.

Antibody staining may include, but is not limited to, H2 antibody, anti-lamellocyte antibody (L1a), or anti-plasmatocyte antibody (P1b). After centrifugation, cells are resuspended in staining medium, and the final antibody solutions are added for an incubation on ice, and in the dark. Controls are incubated with medium only. After incubation, antibody-stained and control cells are washed once with ice-cold staining medium and centrifuged for 5 min at 4° C. at 1,500 rpm. Supernatants are discarded and the pellet was kept on ice in the dark. Just before acquisition on the FACS machine (˜1 h after last incubation step), cell pellets may be resuspended in staining medium with propidium iodide (PI; Sigma) to label dead cells.

Multidimensional analyses and cell sorting may be performed on a modified FACStar Plus® (Pharmingen) equipped with three (3) lasers (i.e., for example, a krypton laser at 407 nm, an argon laser at 488 nm, and a dye laser at 595 nm) and thirteen (13) detectors (i.e., for example, eleven (11) fluorescence plus forward detectors and two (2) side scatter detectors). All analyses and sorts are repeated at least two or three times. The purity of sorted fractions was checked visually and by FACS reanalysis. Images of sorted cell fractions may be obtained with a Eclipse E800® microscope (Nikon). Data can be compensated, analyzed, and presented by using FLOWJO® software (Tree Star, Ashland, Oreg.). FACS machines may be standardized with fluorochrome-containing beads, and fluorescence-reading in each channel automatically adjusted to a constant value to ensure that data obtained on different days were comparable.

B. Enzyme Linked Immunoabsorbent Assay (ELISA)

Most enzyme-immunoassays are analogous to fluorescence or radioimmunoassays in that they involve at least one separation step in which the ‘bound’ enzyme labeled reagent is separated from the unbound enzyme, enabling measurement of either bound or free activity. This is the basis for the ‘enzyme-linked immunosorbent assay’ (ELISA).

ELISA tests can be competitive for assay of an antigen. For example, an enzyme labeled antigen is mixed with a test sample containing antigen, which competes for a limited amount of antibody. The reacted (bound) antigen is then separated from the free material, and its enzyme activity is estimated by addition of substrate. An alternative method for antigen measurement is the double antibody sandwich technique. In this modification a solid phase is coated with specific antibody. This is then reacted with the test sample containing antigen, then enzyme labeled specific antibody is added, followed by the enzyme substrate. The ‘antigen’ in the test sample is thereby ‘captured’ and immobilized on to the sensitized solid phase where it can itself then fix the enzyme labeled antibody. This technique is analogous to the immunoradiometric assays

In an indirect ELISA method, an antigen is immobilized by passive adsorption on to the solid phase. Test sera are then incubated with the solid phase and any antibody in the test sera becomes attached to the antigen on the solid phase. After washing to remove unreacted serum components an antiglobulin enzyme conjugate is added and incubated. This will become attached to any antibody already fixed to the antigen. Washing again removes unreacted material and finally the enzyme substrate is added. Its color change will be a measure of the amount of the conjugate fixed, which is itself proportional to the antibody level in the test sample.

Various other modifications of ELISA have been used. For example, a system where the second antibody used in the double antibody sandwich method is from a different species, and this is then reacted with an anti-immunoglobulin enzyme conjugate. The advantage of this is that it avoids the labeling of the specific antibody, which may be in short supply and of low potency. This same method can be used to assay antibody where only an impure antigen is available; the specific reactive antigens are selected by the antibody immobilized on the solid phase.

In another ELISA assay for antigen, plates are coated with a specific antigen and these are then incubated with a mixture of reference antibodies and a test sample. If there is no antigen in the test sample the reference antibody becomes fixed to an antigen sensitized surface. If there is antigen in the test solution this combines with the reference antibody, which cannot then react with the sensitized solid phase. The amount of antibody attached is then indicated by an enzyme labeled antiglobulin conjugate and enzyme substrate. The amount of inhibition of substrate degradation in the test sample (as compared with the reference system) is proportional to the amount of antigen in the test system.

In all these methods passive adsorption to the solid phase can be used in the first step. Adsorption of other reagents can be prevented by inclusion of wetting agents in all the subsequent washing and incubation steps. Washing should be performed to prevent carry-over of reagents from one step to the next.

C. Immunohistochemistry (IHC)

Immunohistochemistry studies the chemical composition and structure of animal and plant tissues. Microscopic, x-ray diffraction, and/or radioactive tracer techniques may be used. Direct viewing of the cellular distribution of a molecule (marker) may be accomplished by using labeled antibodies or other ligands including nucleic acid probes. Labels include, but are not limited to, enzymes, radioisotopes, and fluorescent molecules. The technique can be applied to whole cells, for example for identification of lymphomas (white blood cell cancers) or to tissue sections, for example for cancer diagnosis.

The specific (or primary) antibody may be labeled directly. Alternatively, and more often, a second antibody carrying the label is used to specifically bind to the first. Also, the tissue may require pretreatment to reveal the marker of interest (i.e., for example, enzymic) or to remove non-specific effects.

A number of methods have been developed to amplify the visual signal and this may add several steps to the technique. Ultimately, in the case of an enzyme-label a substrate is applied which produces a colored product at the site of the label. The surrounding tissue is then counterstained to provide contrast. Therefore, most protocols for carrying out staining involve a large number of incubations of various time periods, separated by washing to remove spent reagents.

Each operation requires considerable care in the application of small amounts (i.e., for example, 50 to 200 microliters) of reagents to cover the tissue adequately and also in the washing steps to ensure complete removal of spent reagents and to avoid accidental removal of tissue from the glass specimen slides. The glass specimen slides are supported on trays. When staining is carried out above ambient temperature, heating is normally provided using a specially designed temperature-controlled template with provision to allow high humidity.

The slides may be washed with a buffer stream from a hand-operated dispensing bottle. The slides can then cleared of liquid by being set vertically to drain, and/or wiped around the specimen with paper towel material.

Biochemical agent(s) delivery is via a manual pipettor positioned by eye such that the fluid is spread to cover the tissue sample. The control of event sequences and times may be performed manually or using automated systems that are commercially available.

D. Fluorescence Microscopy

Fluorescence microscopy has long been used as a descriptive adjunct to quantitative biochemical techniques in studies of cellular organization and physiology. In the late 1970s, sensitive imaging detectors became commercially available and gave fluorescence microscopy the potential to be a quantitative tool. However, because of the prohibitive cost and sophistication of high-speed image processing computers, quantitative fluorescence microscopy was generally limited to relatively few laboratories with a specific interest in “digital imaging microscopy.” This situation has changed in the past 10 years with the revolution in digital technology. Inexpensive personal computers are now capable of tasks that once required large mainframe computers. Integrated optical imaging systems are now commercially available that are capable of processing the entire assay from the biological preparation to the final data. In parallel, significant improvements have been made in optical elements and imaging hardware. Sensitive fluorescent indicators of a variety of physiologically important properties have been introduced, and new fluorescent reagents are continually being developed for sensitively and specifically characterizing the intracellular distribution of proteins, nucleotides, ions, and lipids.

As quantitative microscopy becomes more widely available, it is particularly important that researchers new to fluorescence microscopy be aware of the factors that may complicate quantification of fluorescence. The amount of fluorescence detected is affected by the properties of illumination sources, the optical and spectroscopic properties of the microscope, and the resolution, sensitivity, and signal-to-noise properties of the detector. Fluorescence emissions are attenuated by the photobleaching that accompanies illumination. At high concentrations of fluorophore, interactions between fluorophore moieties can alter the amount and/or spectrum of fluorescence emissions. For certain fluorophores, fluorescence is also sensitive to the immediate physical environment (i.e., for example, ionic composition) of the fluorophore.

E. Ratio Fluorescence Microscopy

In ratio fluorescence microscopy two fluorescence images are collected and the parameter of interest is quantified as a ratio of the fluorescence in one image to that in the other image. For example, ratio fluorescent ion indicator include, but are not limited to, fluorescein and fura-2, whose excitation spectra change shape upon binding protons or calcium ions, respectively. In the case of fluorescein, fluorescence excited by 490 nm light is efficiently quenched by proton binding, whereas fluorescence excited by 450 nm light is relatively unaffected. Although the quantity of fluorescein fluorescence emitted by a volume when excited with 490 nm light depends on the pH of that volume, it is also affected by all the other factors listed above as well as by the concentration of fluorescein in the volume. However, the ratio of fluorescence excited by 90 nm light to that excited by 450 nm depends on pH, but is relatively independent of many variables that affect quantification in single wavelength images: fluorophore concentration, photobleaching, lateral heterogeneity in illumination and detector sensitivity, and differences in optical path length. Spectroscopic variation in illumination and detection is circumvented by calibrating the microscopic system with known pH standards.

Fluorescence ratio images may be collected by sequentially exciting the sample with two different wavelengths of light and sequentially collecting two different images, by exciting the sample with a single wavelength of light and collecting images formed from light of two different emission wavelengths, or by exciting the sample with two wavelengths and collecting emissions of two wavelengths. Ion indicators have been developed for both excitation ratio microscopy (i.e., for example, fura-2 for calcium and fluorescein for pH) and for emission ratio microscopy (i.e., for example, indo-1 for calcium and SNARF for pH).

In fluorescence resonance energy transfer (FRET), an excited fluorescent donor molecule, rather than emitting light, transfers that energy via a dipole-dipole interaction to an acceptor molecule in close proximity. If the acceptor is fluorescent, then the decrease in donor fluorescence due to FRET is accompanied by an increase in acceptor fluorescence (i.e., for example, sensitized emission). The amount of FRET depends strongly on distance, typically decreasing as the sixth power of the distance, so that fluorophores can directly report on phenomena occurring on the scale of a few nanometers, well below the resolution of optical microscopes. Among other things, FRET has been used to map distances and study aggregation states, membrane dynamics, or DNA hybridization.

In principle, FRET measurements can provide information about any system whose components can be manipulated to change the proximity of donors and acceptors on the scale of a few nanometers. In practice, the ability to label a system of interest with appropriate donors and acceptors is constrained by several physical and instrumental factors. In addition to the requirement that donor and acceptor be in close proximity, the donor emission and acceptor absorption spectra should overlap significantly with minimal overlap of the direct excitation spectra of the two fluorophores. Instrumental differences between a fluorescence microscope and a spectrofluorometer, i.e., spatial confinement of the signal, reduced sensitivity, and generally limited wavelength selection, all affect the quality and quantity of information that can be extracted from a FRET experiment using a microscope. The use of FRET in its traditional incarnation as a molecular ruler to measure absolute distances is often not feasible in the fluorescence microscope. Rather, FRET ratio imaging microscopy is often used as an indicator of proximity, subject to some degree of calibration.

The simplest experimental approach is to excite the donor and measure both the direct donor emission “DD” and the sensitized emission “DA” of the acceptor (the first letter represents the species being excited, and the second letter represents the observed emission). The ratio of acceptor-to donor fluorescence, DA/DD, varies between two extremes: no energy transfer and maximal energy transfer. When donor and acceptor are sufficiently distant, no energy transfer occurs and the donor fluorescence (DD) is at its maximum, whereas the sensitized emission is zero. Acceptor fluorescence results only from direct excitation of the acceptor, and DA/DD is at its minimum. The greatest amount of energy transfer occurs when the donor and acceptor are separated by the shortest possible distance, and excited donors lose most of their energy to the acceptor.

Complete quantification of FRET can involve significant calculations, but an estimation of FRET can be obtained easily by measuring the intensity at two fixed time points and taking the ratio of these intensities.

To quantify the relative amount of an acceptor, the acceptor can also be excited directly with the wavelength ideal for acceptor fluorescence, so that “AA” is recorded rather than DA. With AA used as the reference, the ratio DD/AA can also be used as a measure of FRET. Measurement of AA does not generally affect the measurement of DD because acceptor excitation wavelengths are always longer (lower energy) than donor excitation wavelengths, thus avoiding photobleaching of the donor.

Although photobleaching should usually be minimized, it can in some cases actually be exploited to measure FRET. Photobleaching of the donor usually occurs when it is in the excited state: before fluorescence emission occurs there is some probability that photobleaching will remove that fluorophore from the excited state, and also from future excitation emission cycles. When FRET occurs, the donor is removed from the excited state before emission or photobleaching, and the bleach rate decreases because that donor remains available for another cycle of excitation emission. The efficiency of FRET can be determined from the bleach rate of donor fluorescence in the presence of acceptor compared with the bleach rate of the donor in the absence of acceptor. Experimentally, the instantaneous intensity, 1(t), is normalized to the initial intensity 1(0) and the decay of fluorescence intensity is analyzed. A major advantage of the photobleaching method is that it uses only a single excitation wavelength and only a single emission wavelength. The bleach rate of the donor in the absence of acceptor should be measured under experimental conditions identical to those for the donor-acceptor pair, because bleaching rates can vary significantly for different intracellular environments.

If: i) the amount of FRET is relatively small; ii) the acceptor is not fluorescent; or iii) rapid photobleaching prevents measurement of static fluorescence intensities, a photobleaching method may provide the only practical measurement of FRET. In particular, the photobleaching method should be useful with the high illumination intensities typical with lasers used for confocal microscopy.

F. Western Blot

Purified proteins can be immunoblotted onto nitrocellulose membranes (0.45-μm pore size; Bio-Rad). All sera may be screened at a dilution of 1:100, followed by incubation with, for example, a peroxidase-conjugated secondary antibody according to the manufacturer's instructions. (Dako, Glostrup, Denmark). 3,3′-diaminobenzidine tetrahydrochloride (DAB; Pierce, Rockford, Ill.) may be used as a horseradish peroxidase substrate for membrane color development.

In one embodiment, the present invention contemplates a method comprising an immunofluorescence assay. For example, a phage may be propagated in cells at 37° C. until a monolayer confluence is formed. After which, the cells were harvested, spotted onto Teflon-coated slides, and fixed with 80% cold acetone. Samples may be tested at a 1:10 dilution and washed with 1×PBS after being incubated either for 90 min, followed by incubation with a fluorescein isothiocyanate (FITC)-conjugated rabbit anti-human immunoglobulin M (IgM), or for 30 min, followed by incubation with a FITC-conjugated anti-human IgG, and then incubated further at 37° C. The slides can be subjected to another washing cycle before being monitored for specific fluorescence under an immunofluorescence microscope. The sensitivity and specificity of a Western blot assay may be calculated, for example, by using the following equations: sensitivity: number of true positive samples/(number of true positive samples+number of false negative samples)×100; and specificity: number of true negative samples/(number of true negative samples+number of false positive samples)×100.

G. In Vivo Imaging

In one embodiment, the present invention contemplates in vitro and in vivo optical imaging methods for assessing activity of an agent. In one embodiment, a small high affinity peptide/fluorochrome binding pair may be used to assess molecular targets in cell culture and/or in living animals, such as humans.

Light in the visible-wavelength range is routinely used for conventional and intravital microscopy. Because hemoglobin (i.e., for example, an absorber of visible light), or water and lipids (i.e., for example, absorbers of infrared light) have their lowest absorption coefficient in the near-infrared (NIR) region of approximately 650-900 nm, the use of NIR light is ideal for imaging deeper tissues. Although it is not necessary to understand the mechanism of an invention, it is believed that imaging in the NIR spectrum (700-900 nm) maximizes tissue penetrance in addition to minimizing the autofluorescence from non-target tissue.

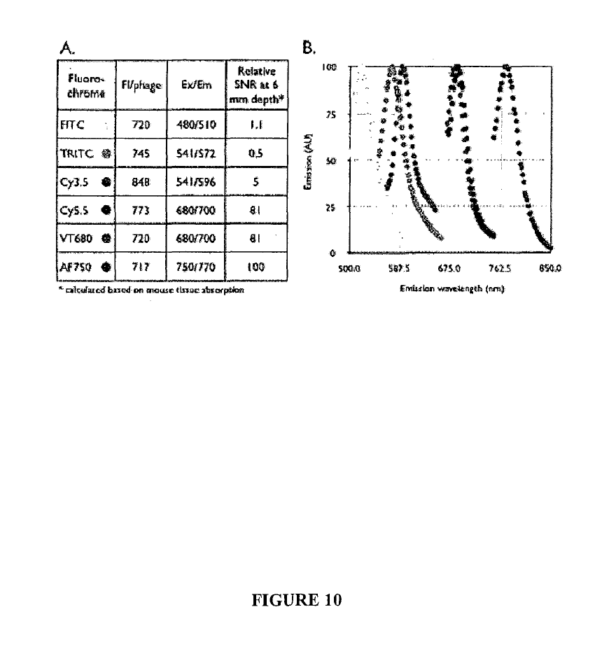

Fluorochromes are available with different spectral properties that allow multicolor imaging (i.e., fore example, measurements are made using a control reference channel) and/or simultaneous screening of differently labeled phages. FIG. 10. Multicolor imaging and/or simultaneous phage screening can rapidly eliminate clones that would ultimately fail because of unfavorable in vivo pharmacokinetics, delivery barriers, or insufficient target-to background ratios.

NIR fluorescence imaging relies on light with a defined bandwidth as a source of photons that encounter a fluorescent molecule (i.e., for example, an optical contrast agent). This fluorescent molecule then emits a signal with different spectral characteristics that can be resolved with an emission filter and captured with an ultrasensitive charge-coupled device (CCD) camera. Interpretation of NIR data and images generally requires advanced data processing techniques to account for the diffuse nature of photon propagation in tissue.

The recent advances in NIRF imaging have been accelerated by the development of NIR fluorochromes coupled to quenching peptides that are activated by specific proteases at the target site. Becker et al., “Receptor-targeted optical imaging of tumors with near-infrared fluorescent ligands” Nat Biotechnol 19:327-331 (2001). NIRF probes may have a delivery vehicle (i.e., for example, a targeting moiety) linked to a near infrared fluorochrome. Delivery vehicles are long-circulating, high-molecular weight synthetic-protected graft copolymers that have already been tested in clinical trials. Callahan et al., “Preclinical evaluation and phase 1 clinical trial of a ⁹⁹mTc labeled synthetic polymer used in blood pool imaging” Am J Roentgenol 171:137-143 (1998). Although it is not necessary to understand the mechanism of an invention, it is believed that most disease processes have a molecular basis that can be exploited to detect disease earlier or to monitor novel therapies by imaging molecular biomarkers.

For example, studies with NIRF chromophores, both in culture and in vivo, have shown that chromophores having a very low background fluorescence can increase the fluorescence over several hundredfold with chromophores detectable in the nanomolar range and with no apparent toxicity. Weissleder et al., “Molecular imaging” Radiology 219:316-333 (2001); and Yu et al., “Novel targets for directed cancer therapy” Drugs Aging 11: 229-244 (1997). In the past few years, NIRF imaging aided by activatable NIRF probes has been used for example in detecting tumors, apoptosis, and other molecular events in experimental models.

Optical imaging of tissues (i.e., for example, cancer tissue) in animal models offers many potential advantages including, but not limited to, rapid imaging, lack of contrast agents, radiation and substrates. However, optical imaging has been challenging for cancer cells because these cells usually do not have a specific optical quality that clearly distinguishes them from normal tissue. Also, conventional optical imaging has been severely limited by the strong absorbance and scattering of the illuminating light by tissue surrounding the target. As a result, neither the sensitivity nor spatial resolution of current methods are sufficient to image early stage tumor growth or metastasis. Previous attempts to endow tumor cells with specific, detectable spatial markers have mostly met with indifferent success. These included labeling with monoclonal antibodies and other high-affinity vector molecules targeted against tumor-associated markers. However, results were limited due to achieving only a low tumor/background contrast and to the toxicity of the procedures. Optical imaging may be optimized by making a target tumor cell the source of light. This renders the incident light scattering much less relevant. A high affinity attachment of a fluorochrome to a tumor cell provides the necessary sensitivity and specificity to convert a cell into a light source.

In one embodiment, the present invention contemplates using a small high affinity peptide/fluorochrome binding pair to image apoptotic cells. Apoptosis is believed to comprise programmed cell death and is involved in such diseases including, but not limited to, autoimmune disorders, organ and bone marrow transplant rejection, or cancer. Although it is not necessary to understand the mechanism of an invention, it is believed that the transformation of a normal cell to an apoptotic cell is typically characterized by loss of cell volume, plasma membrane blebbing, nuclear chromatin condensation and aggregation, and endonucleocytic degradation of DNA into nucleosomal fragments. Reed J C., “Apoptosis-based therapies” Nat Rev Drug Disc 1:111-121 (2002). Further, it is believed that these cell changes occur following sequential activation of initiator and effector caspases (cysteinyl aspartate-specific proteinase). Kim et al., “Cellular non-heme iron content is a determinant of nitric oxide-mediated apoptosis, necrosis, and caspase inhibition” J Biol Chem 275:10954-10961 (2000).

Caspase-1, also known as interleukin 1β-converting enzyme (ICE), is considered an initiator caspase. When using a NIRF probe selective for caspase-1 (ICE), apoptosis can be detected when induced by compounds including, staurosporine, ganciclovir, ionizing radiation or by infection of cells with herpes simplex virus amplicon vector (HSV-ICE-lacZ). Messerli et al., “A novel method for imaging apoptosis using a caspase-1 (ICE) near-infrared imaging probe” Neoplasia 6:95-105 (2004). Thus, ICE-NIRF probe can be used in monitoring endogenous and vector-expressed caspase-1 activity in cells and should prove useful in monitoring endogenous and vector-expressed caspase-1 activity, and potentially apoptosis in cell culture and in vivo.

One of the earliest indications of apoptosis may be the translocation of the membrane phospholipid phosphatidylserine (PS) from the inner to the outer leaflet of the plasma membrane. Although it is not necessary to understand the mechanism of an invention, it is believed that once exposed to the extracellular environment, Annexin V binding sites on PS are exposed. Annexin V is believed a Ca²⁺-dependent, phospholipid binding protein with affinity for PS. A NIRF annexin V probe may be used for optical sensing of tumor apoptotic environments. Such a probe specificity was assessed using nude mice each bearing a cyclophosphamide (CPA) chemosensitive Lewis lung carcinoma (LLC) and a chemoresistant LLC(CR-LLC). After injection with active annexin V, the LLC of CPA-treated mice had significant elevations of the tumor-annexin V ratio (TAR; tumor NIRF/background NIRF), but only a moderate increase was obtained for the CR-LLC indicating that active Cy-annexin V and surface reflectance fluorescence imaging provide a nonradioactive, semiquantitative method of determining chemosensitivity in LLC xenografts. Schellenberger et al., “Optical imaging of apoptosis as a biomarker of tumor response to chemotherapy” Neoplasia 5:187-192 (2003).

In one embodiment, the present invention contemplates a method comprising a small high affinity peptide (i.e., for example, IQPSHFF (SEQ ID NO: 1))/fluorochrome binding pair, wherein the binding pair is targeted to a tumor cell.

Fluorescence imaging may be accomplished by, for example, a Leica fluorescence stereo microscope model LZ12 equipped with a mercury 50 W lamp power supply. To visualize to different fluorescence sources (i.e., for example, green fluorescent protein (GFP) and red fluorescent protein (RFP)) at the same time, excitation may be produced through a D425/60 band pass filter, 470 DCXR dichroic mirror and emitted fluorescence may be collected through a long pass filter GG475 (Chroma Technology, Brattleboro, Vt., USA). Macroimaging can then be carried out in a light box (Lightools Research, Encinitas, Calif., USA). Fluorescence excitation of both GFP and RFP probes can be produced through an interference filter 440±20 nm using slit fiber optics for illumination. Fluorescence is observed through a 520 nm long pass filter. Images from the microscope and light box are captured on a Hamamatsu C5810 3-chip cool color CCR camera (Hamamatsu Photonics Systems, Bridgewater, N.J., USA). 19 Images can be processed for contrast and brightness and analyzed with the use of Image Pro Plus 4.0 software (Media Cybernetics, Silver Springs, Md., USA). High resolution images of 1024×724 pixels are captured directly on an IBM PC or continuously through video output on, for example, a high resolution Sony VCR model SLV—R1000 (Sony Corp., Tokyo, Japan).

### V. Cell Expression of Peptide/Chromophore Binding Pairs

In one embodiment, the present invention contemplates a cell comprising a small high affinity peptide/chromophore binding pair wherein the cell is expressing the high affinity peptide. In one embodiment, the binding pair is attached to the cell surface by a targeting moiety, wherein the targeting moiety is selected from the group comprising an antibody or antibody fragment, a receptor or a ligand. In one embodiment, the cell is transfected with a heterologous genetic construct encoding the high affinity peptide and the cell is expressing the high affinity peptide. In one embodiment, the cell is prokaryotic. In one embodiment, the cell is eukaryotic. In one embodiment, the present invention contemplates an expression vectors comprising a plurality of regulatory elements, wherein the vectors are capable of encoding a high affinity peptide. In one embodiment, the vector comprises a promoter. In one embodiment, the promoters are commercially available and may be selected from the group comprising CMV (cytomegalovirus), SV40 (Simian Virus 40) or an inducible promoter such as the Tet System® and the Ecdysone-Inducible Expression System® (with Ponasterone A) (both available from Invitrogen, Inc.). In one embodiment, binding pair constructs are transiently transfected into a cell type, including, but not limited to, somatic cells, primary culture cells, or lymphoid cells. In one embodiment, a stable transfectant is established from a cell line. In one embodiment, a cell line includes, but is not limited to, HEK-293T, HeLa, Daudi, K562, or COS cell lines. In one embodiment, a transfected cell line expresses the binding pair.

To obtain high level expression of a cloned gene, such as those cDNAs encoding a high affinity peptide as contemplated herein, it is important to construct an expression vector that contains, at the minimum, a strong promoter to direct transcription, a ribosome binding site for translational initiation, and a transcription/translation terminator. Suitable bacterial promoters are described, e.g., in Sambrook et al. and Ausubel et al. (both references herein incorporated by reference). Bacterial expression systems for expressing the high affinity protein are available in, e.g., E. coli, Bacillus sp., and Salmonella. Palva et al., Gene 22:229-235 (1983); and Mosbach, et al., Nature, 302:543-545 (1983) (both references herein incorporated by reference). Kits for such expression systems are also commercially available; for example, eukaryotic expression systems for mammalian cells, yeast, and insect cells. In particular, the pET23D expression system (Novagen) is a preferred prokaryotic expression system.

A promoter used to direct expression of a heterologous nucleic acid depends on the particular application. The promoter is preferably positioned about the same distance from the heterologous transcription start site as it is from the transcription start site in its natural setting, however, some variation in this distance can be accommodated without loss of promoter function.

In addition to a promoter, an expression vector typically contains a transcription unit or expression cassette that contains all the additional elements required for the expression of a high affinity protein encoding DNA in host cells. A typical expression cassette thus contains a promoter operably linked to the DNA sequence encoding a high affinity protein and signals required for efficient polyadenylation of the transcript, ribosome binding sites, and translation termination. The DNA sequence encoding a high affinity protein may typically be linked to a cleavable signal peptide sequence to promote secretion of the encoded protein by the transformed cell. Such signal peptides would include, but not be limited to, signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, an expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any conventional vector may be used for expression in eukaryotic and/or prokaryotic cells. Standard bacterial expression vectors include, but are not limited to, plasmids such as pBR322 based plasmids, pSKF, and fusion expression systems such as GST and LacZ. Epitope tags can also be added to recombinant proteins to provide convenient methods of isolation. One preferred embodiment of an epitope tag is c-myc.

Expression vectors containing regulatory elements from eukaryotic viruses are typically used in eukaryotic expression vectors, e.g., SV40 vectors, papilloma virus vectors, and vectors derived from Epstein Bar virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a high affinity protein encoding sequence under the direction of the polyhedrin promoter or other strong baculovirus promoters.

The elements that are typically included in expression vectors may also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. Prokaryotic sequences are preferably chosen such that they do not interfere with the replication of the DNA in eukaryotic cells.

Standard transfection methods are used to produce bacterial, mammalian, yeast or insect cell lines that express large quantities of high affinity protein, which are then purified using standard techniques. Colley et al., J. Biol. Chem. 264:17619-17622 (1989); and “Guide to Protein Purification”, In: Methods in Enzymology, vol. 182, Deutscher Ed., (1990) (both references herein incorporated by reference). Transformation of eukaryotic and prokaryotic cells may also be performed according to standard techniques. Morrison, J. Bact., 132:349-351 (1977); and Clark-Curtiss & Curtiss, In: Methods in Enzymology, 101:347-362, Wu et al., Eds, (1983) (both reference herein incorporated by reference).

Many procedures for introducing foreign nucleotide sequences into host cells may be used. These include, but are not limited to, the use of calcium phosphate transfection, polybrene, protoplast fusion, electroporation, liposomes, microinjection, plasma vectors, viral vectors and any of the other method that introduces cloned genomic DNA, cDNA, synthetic DNA or other foreign genetic material into a host cell. Sambrook et al., (herein incorporated by reference; supra). A particular genetic engineering procedure is compatible with the presently contemplated invention if a successful introduction of at least one gene into a host cell capable of expressing a high affinity protein is accomplished.

After the expression vector is introduced into the cells, the transfected cells are cultured under conditions favoring expression of a high affinity protein which is the purified by using one of many standard techniques including, but not limited to, functional assays. A high affinity protein may also be purified to substantial purity by standard techniques including, but not limited to, selective precipitation with such substances as ammonium sulfate; column chromatography, immunopurification methods, and others. Scopes et al., “Protein Purification: Principles and Practice” U.S. Pat. No. 4,673,641 (herein incorporated by reference); Ausubel et al., supra; and Sambrook et al., supra)(both references herein incorporated by reference). A preferred method of purification comprises using Ni-NTA agarose (Qiagen).

A number of procedures can be employed when a recombinant high affinity protein is being purified. For example, proteins having established molecular adhesion properties can be reversibly fused to a high affinity protein. With an appropriate ligand, a high affinity protein can be selectively adsorbed to a purification column and then freed from the column in a relatively pure form. The fused protein is then removed by enzymatic activity. Further, a high affinity protein could be purified using immunoaffinity columns.

Recombinant proteins are usually expressed by transformed bacteria in large amounts, typically after promoter induction; but expression can be constitutive. Bacteria are grown according to standard procedures in the art. For high affinity proteins that may be difficult to isolate with an intact biological activity, fresh bacteria cells are used for isolation of these types of proteins. Use of cells that are frozen after growth but prior to lysis typically results in negligible yields of active protein.

Proteins expressed in bacteria may form insoluble aggregates (“inclusion bodies”). Several protocols are suitable for purification of high affinity protein inclusion bodies. For example, purification of inclusion bodies typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be homogenized using a Polytron (Brinkman Instruments, Westbury, N.Y.). Alternatively, the cells can be sonicated on ice. Alternate methods of lysing bacteria are apparent to those of skill in the art. Sambrook et al., supra; Ausubel et al., supra (both reference herein incorporated by reference).

A cell suspension may be centrifuged and a resulting pellet containing the inclusion bodies resuspended in buffer, that does not dissolve, but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, inclusion bodies may be solubilized by the addition of a solvent that is both a strong hydrogen acceptor and a strong hydrogen donor (or a combination of solvents each having one of these properties); the proteins that formed the inclusion bodies may then be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (i.e., for example, by dialysis) or dilution of the denaturant, allowing re-formation of immunologically and/or biologically active protein. After solubilization, the protein can be separated from other bacterial proteins by standard separation techniques.

Alternatively, it is possible to purify a high affinity protein from bacteria periplasm. Where a high affinity protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO.sub.4 and kept in an ice bath for approximately 10 minutes. The cell suspension is centrifuged and the supernatant decanted and saved.

Then recombinant proteins present in the supernatant can be separated from the host proteins by standard separation techniques. One such technique is solubility fractionation. Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Another standard separation technique utilizes size differential filtration. For example, when a high affinity protein has a known molecular weight, this knowledge can be used to isolated it from proteins of greater and/or lesser size using ultrafiltration through membranes of different pore size (for example, Amicon or Millipore membranes). As a first step, the protein mixture may be ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate.

Alternatively, a protein can be separated from other proteins by column chromatography where separation is based upon differences in size, net surface charge, hydrophobicity, and affinity for ligands. In addition, antibodies raised against proteins can be conjugated to column matrices and the proteins immunopurified. It will be apparent to one of skill that chromatographic techniques can be performed at any scale and using equipment from many different manufacturers (e.g., Pharmacia Biotech).

### VI. Physiological Disease States

The methods of the invention can also be used in the detection, characterization (i.e., for example, genotype and/or phenotype) and/or determination of the localization of a disease, the severity of a disease or a disease-associated condition. Examples of such disease or disease-conditions include, but are not limited to, inflammation (i.e., for example, inflammation that results in arthritis, such as rheumatoid arthritis), all types of cancer, cardiovascular disease (i.e., for example, atherosclerosis and inflammatory conditions of blood vessels), dermatologic disease (i.e., for example, Kaposi's Sarcoma, psoriasis), ophthalmic disease (i.e., for example, macular degeneration and diabetic retinopathy), infectious disease, immunologic disease (i.e., for example, Acquired Immunodeficiency Syndrome, lymphoma, type I diabetes, and multiple sclerosis), neurodegenerative disease (i.e., for example, Alzheimer's disease), and bone-related disease (i.e., for example, osteoporosis and primary and metastatic bone tumors).

A number of tumors have been shown to have elevated levels of proteolytic enzymes at an early stage. The presence of these enzymes represents an attractive target for tumor imaging and designing therapeutic strategies. Yu et al., “Novel targets for directed cancer therapy” Drugs Aging 11:229-244 (1997). Using this as a basis, autoquenched NIRF probes that become active after protease activation have been used in imaging tumors that have upregulated levels of certain proteases, like cathepsins. Cathepsin B and cathepsin H protease activities have also been used to detect submillimeter sized tumors using NIR fluorescent probes. Weissleder et al., “In vivo imaging of tumors with protease-activated near-infrared fluorescent probes” Nat Biotech 17:375-378 (1999).

Similar to cathepsins, matrix metalloproteinases (MMPs) are overexpressed in a number of tumors, and inflamed tissues. Davies et al., “Levels of matrix metalloproteases in bladder cancer correlate with tumor grade and invasion” Cancer Res 53:5365-5369 (1993); Zucker et al., “Measurement of matrix metalloproteinases and tissue inhibitors of metalloproteinases in blood and tissues. Clinical and experimental applications” Ann NY Acad Sci 878:212-227 (1999); and Black et al., “A metalloproteinase disintegrin that releases tumour-necrosis factor-a from cells” Nature 385:729 (1997), respectively. One problem in assessing the efficacy of such antitumor drugs has been the inability to detect or image antiproteinase activity directly and noninvasively in vivo. Recent developments allow NIRF-MMP substrates to be used as activatable NIRF reporter probes to monitor MMP activity in intact tumors. Bremer et al., “In vivo molecular target assessment of matrix metalloproteinase inhibition” Nat Med 7: 743-748 (2001).

The methods of the invention can therefore be used, for example, to determine the presence of tumor cells and localization of tumor cells, the presence and localization of inflammation, the presence and localization of vascular disease including areas at risk for acute occlusion (vulnerable plaques) in coronary and peripheral arteries and regions of expanding aneurysms, and the presence and localization of osteoporosis. The methods can also be used to follow therapy for such diseases by imaging molecular events modulated by such therapy, including, but not limited to, determining efficacy, optimal timing, optimal dosing levels (including for individual patients or test subjects), and synergistic effects of combinations of therapy.

A number of animal models mimic the progression and symptoms of several different human diseases. For example, animal models for multiple sclerosis, congestive heart failure, Alzheimer's disease, and Parkinson's disease have been established. Smith et al., J. Pharmacol. Toxicol. Methods 43(2):125 (2000); Hilliard et al., J. Immunol. 166(2):1314 (2000); and Yamada et al., Pharmacol. Ther. 88(2):93 (2000). Recombinant technology has provided disease models using transgenic and gene knockout models (i.e., for example, transgenic mice for breast cancer). Hutchinson et al., Oncogene 19(53):6130 (2000).

Membrane translocation signals can also be added to the small high affinity peptide/chromophore binding pair to improve deliverability. Since cellular entry may occur through fluid phase endocytosis, improvement of cellular uptake and assurance of cytoplasmic deposition of the binding probes can be achieved by attaching membrane translocation (or transmembrane) signal sequences. These signal sequences can be derived from a number of sources including, without limitation, viruses and bacteria. For example, a Tat protein-derived peptide having the sequence -Gly-Arg-Lys-Lys-Arg-Arg-Gln-Arg-Arg-Arg-Lys-Gly-Asp-Glu-Val-Asp-Gly-Cys-NH2 (SEQ ID NO:7) may be efficiently internalized into cells. Alternatively, the sequences Gly-Arg-Lys-Lys-Arg-Gln-Arg-Arg (SEQ ID NO:8) and/or Gly-Arg-Lys-Lys-Arg-Arg-Gln-Arg-Arg (SEQ ID NO:9) can also be used.

Other targeting and delivery approaches can also be used such as folate-mediated targeting, liposomes, transferrin, vitamins, carbohydrates and the use of other ligands that target internalizing receptors, including, but not limited to, somatostatin, nerve growth factor, oxytocin, bombesin, calcitonin, arginine vasopressin, angiotensin II, atrial natriuretic peptide, insulin, glucagons, prolactin, gonadotropin, and various opioids. In addition, other ligands can be used that upon intracellular delivery, undergo an enzymatic conversion that leaves the resulting conversion product trapped within the cell, such as nitroheteroaromatic compounds that are irreversibly oxidized by hypoxic cells.

### VII. IQ-Tag Transgenic Animals

In one embodiment, the present invention contemplates a transgenic animal whose genome comprises a transgene encoding a peptide comprising a high affinity binding site to a fluorochrome. In one embodiment, the fluorochrome comprises a benzindol fluorochrome. In one embodiment, the transgenic animal further comprises a diseased tissue. In one embodiment, the genome is derived from the diseased tissue. In one embodiment, the transgene is stably integrated into the genome of the transgenic animal. In one embodiment, the peptide is expressed by the transgene. In one embodiment, the peptide is displayed on the surface of the diseased tissue. In one embodiment, the peptide comprises the amino acid sequence IQSPHFF (SEQ ID NO:1). In one embodiment, the peptide comprises the amino acid sequence HHSHRHH (SEQ ID NO: 16).

In one embodiment, the present invention contemplates a method of making a transgenic animal, comprising: a) providing; i) a vector comprising a promoter operably linked to a nucleic acid sequence encoding a peptide, wherein the peptide comprises a high affinity binding site to a benzindol fluorochrome; ii) an animal comprising a tissue having at least one cell, wherein the cell is capable of undergoing stable transfection by the vector; and b) contacting the vector with the cell under conditions such that the cell becomes transfected with the vector. In one embodiment, the transfection results in a stable integration of the vector. In one embodiment, the promoter is selected from the group comprising a CMV promoter, a SV40 promoter, a metallothionein promoter, a murine mammary tumor virus promoter, a Rous sarcoma virus promoter, or a polyhedrin promoter.

The present invention provides a number of transgenic animals. In one embodiment, transgenic animals are provided which express any nucleic acid sequence encoding a peptide comprising fluorochrome high affinity binding site in selectively targeted diseased cells. These animals provide useful models for the diagnosis and prognosis of specific disease states (i.e., for example, cancer), monitoring and/or identification of therapeutic compounds (i.e., for example, anti-cancer drugs) and identification of genes which play a role in disease progression of various tissues including, but not limited to those in the liver, kidney, epithelia, pancreas, stomach, intestine, trachea, esophagus, colon, epidermis, anus/rectum, lymph nodes, spleen, lung, or cervix.

A. Creating Transgenic Animals

In another embodiment, a construct may be produced in which the PDX Cre lox promoter is an upstream sequence from the open reading frames of a peptide comprising a high affinity binding site for a fluorochrome (i.e., for example, IQSPHFF (SEQ ID NO: 1). This construct may be used to generate transgenic mice which express high affinity binding site peptide mRNA in a tissue-specific appropriate manner. Thus, full-length and spliced high affinity binding site mRNA may be expressed in any transfected tissue. Moreover, high affinity binding site peptide mRNA expression may be localized in one tissue and not in another tissue.

For example, mice comprising a PDX-1 Cre lox construct may be maintained on a C57B/L6 and 129Sve/V mixed genetic background. The presence of a Cre transgene may be detected by PCR using Crel (5′-CCTGTTTTGCACGTTCA CCG) and Cre3 (5′-ATGCTTCTGTCCGTTTGCCG) primers (300-bp band). F1 and F2 animals may be obtained by mating adequate transgenic lines.

A first step in the generation of the transgenic animals of the invention is the introduction of a construct containing the desired heterologous nucleic acid sequence under the expression control of promoter upstream sequences of the invention into target cells. Several methods are available for introducing the expression vector which contains the heterologous nucleic acid sequence into a target cell, including microinjection, retroviral infection, and implantation of embryonic stem cells. These methods are discussed as follows.

1. Microinjection Methods

Direct microinjection of expression vectors into pronuclei of fertilized eggs is a technique for introducing heterologous nucleic acid sequences into the germ line (Palmiter (1986) Ann. Rev. Genet. 20:465-499). Technical aspects of the microinjection procedure and important parameters for optimizing integration of nucleic acid sequences have been previously described (Brinster et al., (1985) Proc. Natl. Acad. Sci. USA 82:4438-4442; Gordon et al., (1983) Meth. Enzymol. 101:411-433; Hogan et al., (1986) Manipulation of the Mouse Embryo: A Laboratory Manual. Cold Spring Harbor, N.Y.: Cold Spring Harbor Lab.).

Once the expression vector has been injected into the fertilized egg cell, the cell is implanted into the uterus of a pseudopregnant female and allowed to develop into an animal. Of the founder transgenic animals born, 70% carry the expression vector sequence in all of their cells, including the germ cells. The remaining 30% of the transgenic animals are chimeric in somatic and germ cells because integration of the expression vector sequence occurs after one or more rounds of replication. Heterozygous and homozygous animals can then be produced by interbreeding founder transgenics. This method has been successful in producing transgenic mice, sheep, pigs, rabbits and cattle (Jaenisch (1988) supra; Hammer et al., (1986) J. Animal Sci.:63:269; Hammer et al., (1985) Nature 315:680-683; Wagner et al., (1984) Theriogenology 21:29).

2. Retroviral Methods

Retroviral infection of preimplantation embryos with genetically engineered retroviruses may also be used to introduce transgenes into an animal cell. For example, blastomeres have been used as targets for retroviral infection (Jaenisch, (1976) Proc. Natl. Acad. Sci USA 73:1260-1264). Transfection is typically achieved using a replication-defective retrovirus carrying the transgene (Jahner et al., (1985) Proc. Natl. Acad. Sci. USA 82:6927-6931; Van der Putten et al., (1985) Proc. Natl. Acad Sci USA 82:6148-6152). Transfection is obtained, for example, by culturing eight-cell embryos, from which the zona pellucida has been removed with fibroblasts which produce the virus (Van der Putten (1985), supra; Stewart et al., (1987) EMBO J. 6:383-388). The transfected embryos are then transferred to foster mothers for continued development. Alternatively, infection can be performed at a later stage. Virus or virus-producing cells can be injected into the blastocoele (Jahner et al., (1982) Nature 298:623-628). Yet another alternative method involves intrauterine retroviral infection of the midgestation embryos (Jahner et al. (1982), supra).

3. Embryonic Stem Cell Implantation

Another method of introducing transgenes into the germ line involves using embryonic stem (ES) cells as recipients of the expression vector. ES cells are pluripotent cells directly derived from the inner cell mass of blastocysts (Evans et al., (1981) Nature 292:154-156; Martin (1981) Proc. Natl. Acad Sci. USA 78:7634-7638; Magnuson et al., (1982) J. Embryo. Exp. Morph. 81:211-217; Doetchman et al., (1988) Dev. Biol. 127:224-227), from inner cell masses (Tokunaga et al., (1989) Jpn. J. Anim. Reprod. 35:113-178), from disaggregated morulae (Eistetter, (1989) Dev. Gro. Differ. 31:275-282) or from primordial germ cells (Matsui et al., (1992) Cell 70:841-847; Resnick et al., (1992) Nature 359:550-551). Expression vectors can be introduced into ES cells using any method which is suitable for gene transfer into cells, e.g., by transfection, cell fusion, electroporation, microinjection, DNA viruses, and RNA viruses (Johnson et al., (1989) Fetal Ther. 4 (Suppl. 1):28-39).

The advantages of using ES cells include their ability to form permanent cell lines in vitro, thus providing an unlimited source of genetic material. Additionally ES cells are the most pluripotent cultured animal cells known. For example, when ES cells are injected into an intact blastocyst cavity or under the zona pellucida, at the morula stage embryo, ES cells are capable of contributing to all somatic tissues including the germ line in the resulting chimeras.

Once the expression vector has been introduced into an ES cell, the modified ES cell is then introduced back into the embryonic environment for expression and subsequent transmission to progeny animals. The most commonly used method is the injection of several ES cells into the blastocoel cavity of intact blastocysts (Bradley et al., (1984) Nature 309:225-256). Alternatively, a clump of ES cells may be sandwiched between two eight-cell embryos (Bradley et al., (1987) in “Teratocarcinomas and Embryonic Stem Cells: A Practical Approach,” Ed. Robertson E. J. (IRL, Oxford, U.K.), pp. 113-151; Nagy et al., (1990) Development 110:815-821). Both methods result in germ line transmission at high frequency.

Target cells which contain the heterologous nucleic acid sequences are recovered, and the presence of the heterologous nucleic acid sequence in the target cells as well as in the animal is accomplished as described supra.

4. Tissue Specific and Cell Type Specific Expression

Selective expression of the gene of interest in tissues and cells of transgenic animals may be determined using several methods. For example, expression of mRNA encoded by the gene of interest may be determined by using in situ hybridization. This involves synthesis of an RNA probe which is specific for a portion of (or the entire) gene of interest, e.g. by using PCR. The PCR amplified fragment is subcloned into a plasmid (e.g., pbluescript (Stratagene)) and the RNA probe synthesized using labelled UTP (e.g. ³⁵S-UTP) and RNA polymerase (e.g., T3 or T7 polymerase (Promega)). Paraffin-embedded tissue sections are mounted on slides, deparaffinized, rehydrated and the protein digested (e.g., with proteinase K), then dehydrated prior to hybridization with the RNA probe at the desired hybridization stringency. Slides are then developed for autoradiography using commercially available developers. Labelling of tissues and cells as detected on the autoradiographs indicates expression in those tissues and cells of the mRNA encoded by the gene of interest. Alternatively, mRNA encoded by the gene of interest may be detected by reverse transcription polymerase chain reaction (RT-PCR).

Alternatively, expression of the protein product of the gene of interest may be determined using immunohistochemical techniques. Briefly, paraffin-embedded tissue sections are dewaxed, rehydrated, treated with a first antibody which is specific for the polypeptide product of the gene of interest. Binding is visualized, for example, by using a secondary biotinylated antibody which is specific for the constant region of the primary antibody, together with immunoperoxidase and 3,3′-amiobenzidine as a substrate. Sections may then be stained with hematoxylin to visualize the cellular histology. Antibody binding of tissues and cells which is detected by antibody binding demonstrates expression of the protein product of the gene of interest in these tissues and cells.

Yet another alternative method for the detection of expression of the protein product of the gene of interest is by Western blot analysis wherein protein extracts from different tissues are blotted onto nitrocellulose filters, and the filters incubated with antibody against the protein product of the gene of interest, followed by detection of antibody binding using any of a number of available labels and detection techniques.

B. Uses for the Transgenic Animals

The transgenic animals of this invention may be used to (a) screen compounds for therapeutic activity, (b) screen compounds for disease inducting activity, (c) identify tissues and/or subcellular regions which play a role in disease progression (i.e., for example, cancer), and (d) provide an in vivo model for disease progression and monitoring.

In using the transgenic animals provided herein to screen potential therapeutic compounds, many compounds currently in use will be screened first to verify the technique because many of their effects are already known. In this situation, the screening process can be used to gather data such as which compounds are most effective at particular disease stages. In addition, compounds which are derivatives of existing efficacious therapeutic compounds, or which have a new mechanism of action may also be administered singly or in combination to determine their effect in altering the incidence, rate of development, or pathology of various disease states.

Another use of the transgenic mice of this invention is to screen potential compounds that are believed to induce diseases. This may be achieved by exposing transgenic animals of this invention, which exhibit peptides having high affinity binding sites to fluorochromes to agents which are suspected of inducing various diseases. These agents are administered either singly or in combination. Where a combination of agents is used, the agents may be administered simultaneously or sequentially.

An additional use of the transgenic animals provided herein is to determine the identity of specific tissues and/or subcellular regions which are involved in the disease progression. This may be done, for example, by mating two different transgenic mice (e.g., a transgenic mouse which expresses a peptide of the present invention in a kidney; and another transgenic mouse which expresses a peptide of the present invention in a pancreas) to produce a double transgenic animal. The double transgenic animal is then used to determine the frequency and rate of development of diseases within each tissue. The identification of conditions which accelerate disease progression in specific tissues provides further targets for therapeutic treatment. Treatment may be accomplished, for example, by administration to the animal of anti-sense nucleotide sequences which target the coding or non-coding regions of selected genes, and/or of antibodies against the polypeptide products of the genes which are known to play a role in disease progression.

A further use of the herein provided transgenic animals is to develop an in vivo model for monitoring disease progression. Most diseases are believed to be associated with specific etiologic agents. It is also believed that genes, as well as hormones, might play a significant role in the development of most diseases. The involvement of hormones, and/or their agonists and antagonists alone or in combination provides a model system in which to induce and monitor the progression of a variety of diseases. This model would then provide a system to screen candidate drugs (as described supra) for their ability to provide therapeutic approaches.

### EXPERIMENTAL

The following examples are intended as illustrative embodiments contemplated by the present invention and should not be interpreted as limiting in any manner.

Cy3.5, Cy5.5 and AF750 were obtained from GE Healthcare (Niskayuna, N.Y.) Vivotag-680 (VT680) and Genhance-680 were obtained from VisEn Medical (Woburn, Mass.). Polystyrene particles were obtained from Bang's Laboratories, INC (Fishers, Ind.). Amino functionalized slides were from Sigma. Omnimax T2 Ultracompetent Bacteria (for transformation and RF-DNA preparations) and custom oligonucleotide primers, were obtained from Invitrogen (Carlsbad, Calif.). Rapid DNA Ligation Kits, which were from Roche (Indianapolis, Ind.). All molecular biology and cloning reagents were from New England Biolabs (Beverly, Mass.). All other chemicals were from Fisher Scientific or Sigma-Aldrich.

### Example 1

### Phage Display Screening for Fluorochrome Binding Peptides

A phage library was used that expresses random 7-amino acid sequences on the N-termini of all five (5) p3 coat proteins. For selection, a model benzindol fluorochrome (VT680) was covalently coupled to BSA and immobilized the VT680-BSA conjugate on Nunc Maxisorp plates (Fisher). In addition, a subtraction well was prepared containing a BSA-only sample. After a subtraction step designed to filter BSA- and/or plastic-binding clones from subsequent pans, four successive rounds of binding, elution, and amplification were performed.

To generate phages comprising IQSPHFF peptide fused to the N-terminus of g7, splice overlap extension PCR was performed using M13KE RFDNA. The following primers were used:

SOE1f:
(SEQ ID NO: 2)
5′-CCATGGGATCCCTCGAGCAGGTCGCGGATTTCGACACAA-
TTTATC-3′ *;
SOE2r:
(SEQ ID NO: 3)
5′-CAAAACTT TAGATCGTTACGCTAACTAT-3′;
SOE3r:
(SEQ ID NO: 4)
5′-CTGCTCGAGGGATCCCATGGTTACTTAGCCGGAACGAGG-
CGCAGACGG-3′;
SOE4f:
(SEQ ID NO: 5)
5′-CTCTTGTTTGCTCCAGACTC-3′;
p7-Nco-IQSf:
(SEQ ID NO: 6)
5′-CCATGGCCATGGCTATTCAGTCTCCTCATTTTTTTG-
GCGGTGGGGAGCAGGTCGCGGATTTCGACACAATTTATC-3′.

Briefly, the final splice overlap product of interest was generated by PCR using excess primers SOE2r and SOE4f and ligated into M13KE3. Plaques containing single phage clones were isolated and screened for the presence of mutant p7 (p7-NBX) by PCR and positive digestion. p7-IQSPHFF-containing phage clones were positively screened by mobility shift in PCR and confirmed by DNA sequencing.

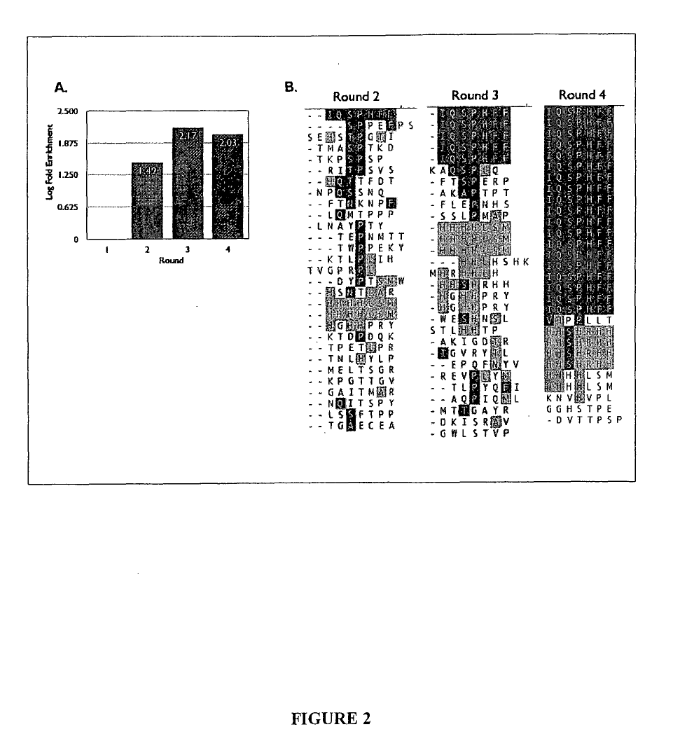

This linear, random seven (7) amino acid phage display library was used to select against VT680, a benzindol fluorochrome. A >100-fold enrichment was observed in the third and fourth rounds of panning, suggestive of a successful selection. FIG. 2A.

Moreover, identification and alignment of amino acid sequences of individual clones from rounds 2, 3, and 4 revealed a robust enrichment and narrowing to a small area of the peptide diversity space. Alignment of the sequences from round 2 showed lack of a single consensus family sequence, however by the third and fourth round, a clear consensus sequence had emerged. FIG. 2B. In the fourth round, 63% (19/30) of the sequences had the sequence IQSPHFF (SEQ ID NO:1) (i.e., referred to herein as IQ-tag).

IQSPHFF (SEQ ID NO:1) would be expected to arise only once in the employed library consisting of 1.28×10⁹ possible 7-residue sequences. This result evinced strong selective pressure in favor of the binding of the affinity sequence. Although rounds two and three did not have clear consensus sequences, the IQSPHFF sequence was present in all rounds with the SP motif appearing in several peptides, further indicating that this motif may be important for binding to benzindol fluorochromes. In addition to the IQSPHFF peptide, a second although less prevalent (20%; 6.30) motif consisting of HHS/HHXH was also identified through the selection.

Notably absent from the list of peptides were “plastic binding” clones whose sequences are abundant in tryptophan and tyrosine, and were previously identified as potential false positive clones during phage display selections. Adey et al., Gene, 156:27-31 (1995).

### Example 2

### Molecular Modeling of Commercially Available Fluorochromes

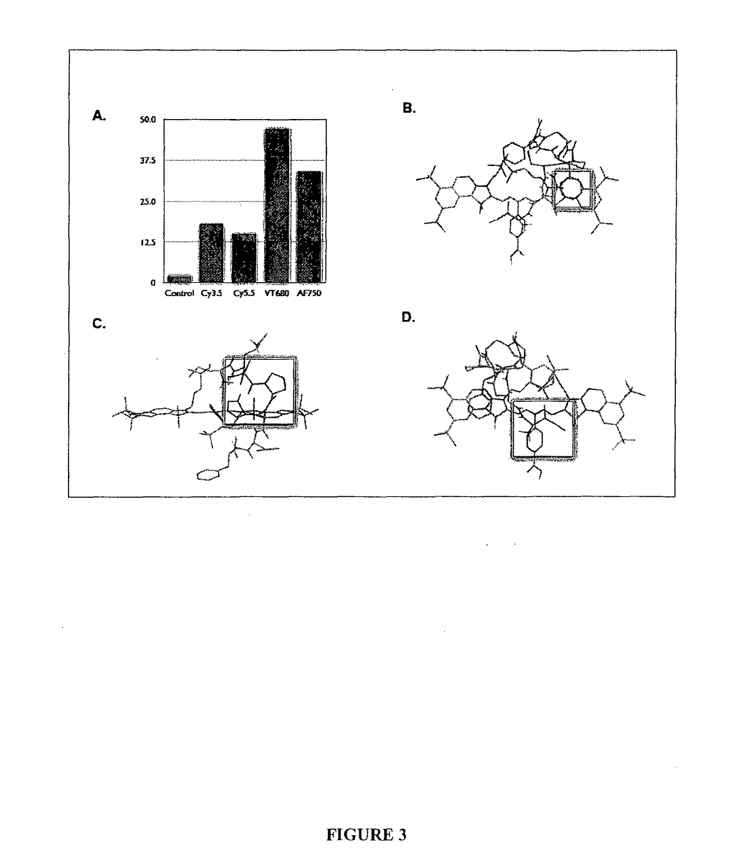

The affinity of IQSPHFF was determined using molecular modeling techniques for different commercially available benzindol fluorochromes (Cy 3.5, Cy 5.5, VT680 and AF750). The highest affinity was to VT680. FIG. 3A. This binding affinity was trailed closely by AF750, whereas Cy3.5 and Cy5.5 both exhibited approximately four-fold less biding than VT680.

To better understand the differences in affinity, molecular modeling was performed, assuming that the fluorochromes were relatively rigid active sites to which the peptide ligand was bound. All molecular modeling, minimization, and docking were accomplished in Cache 6.1 (Fujitsu, Tokyo, Japan). The ground state geometry of the IQSPHFF peptide (SEQ ID NO:1), and each fluorophore, was calculated using CONFLEX/MM3 (medium search) parameters. The peptide was docked onto the fluorophore using the dye as a rigid active site, while the peptide was identified as the flexible ligand. Once docked, the geometry was further optimized using the CAChe FastDock algorithm. All experiments were performed in triplicate to ensure consensus of the binding motif.

In comparing the resulting structures, several additive binding interactions became apparent. The C-terminus of the peptide, containing the hydrophobic and aromatic phenylalanine residues, interacted with one indole or benzoindole subunit of the dye resulting in π-π interactions. FIG. 3B. The next three amino acids, SPH, extended over the alkene linker of the cyanine dyes to the opposite face of the fluorochrome, with the hydrophilic portions directed away from the core. FIG. 3C. Lastly, the hydrophobic N-terminal isoleucine interacted with the similarly hydrophobic alkene linker. FIG. 3D.

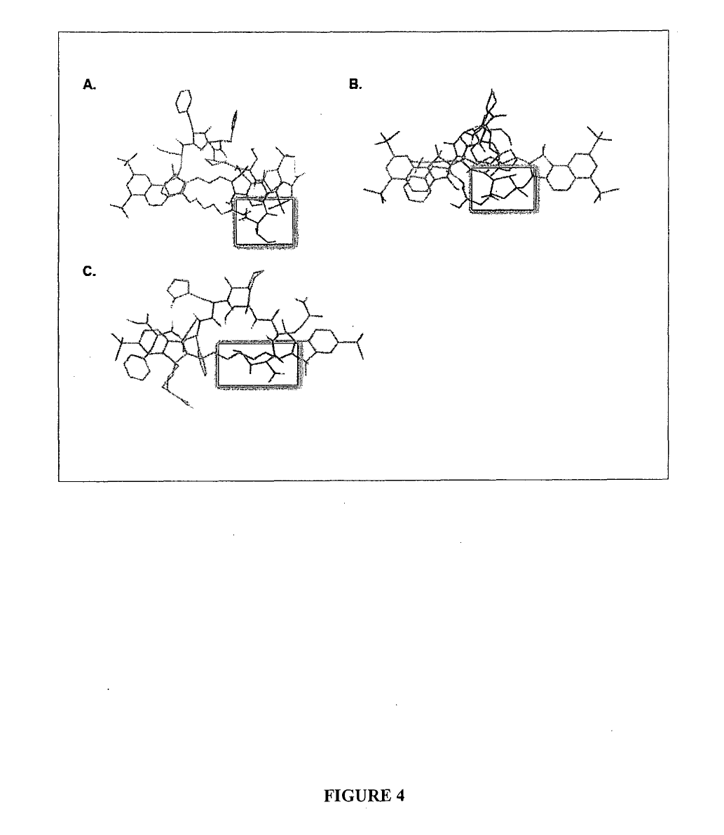

In the case of Cy 3.5, this last interaction was not observed, which is most likely due to the length of the linker and steric strain (the length of the linker in Cy 3.5 is 3 carbons, in Cy 5.5 and VT680 is 5 carbons, and in AF750 is 7 carbons). FIG. 4A. Similar to Cy 3.5, Cy 5.5, VT680 demonstrated a sub-optimal interaction of the isoleucine with the flexible hexanoic acid moiety used in the conjugation of the fluorophore. FIG. 4B. For GH680, all three interactions were present, thereby resulting in optimal docking with the IQSPHFF peptide (SEQ ID NO:1). FIG. 3B-D.

### Example 3

### Binding Affinity & Stoichiometry to a Near Infrared Fluorochrome (NIRF)

The affinity of IQSPHFF to a solid phase bound NIRF (VT680) was determined by generating binding curves forming a classic sigmoidal shape. Specific binding of VT680 to IQSPHFF displayed an average K_{d} of 0.53±0.2 nmol/L.

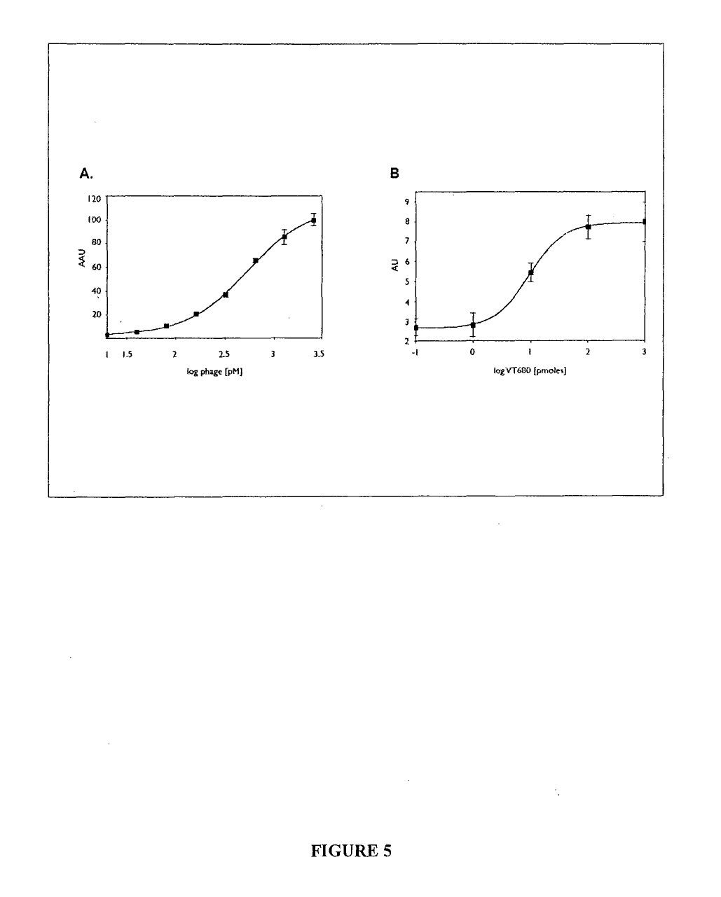

To determine if this binding was specifically due to the presentation of IQSPHFF, a nucleic acid sequence encoding this peptide was synthesized such that it was expressed from phages as an N-terminal extension of a phage p7 coat protein (N′-p7-AIQSPHFF). FIG. 5A. The N′-p7-AIQSPHFF protein likewise demonstrated a sigmoidal dose response binding with identical affinity (K_{d} 0.53±0.12 nmol/L; r²=0.984).

In contrast, phages not expressing display peptides failed to yield convincing sigmoidal concentration response curves and had negligible binding. These results indicate that binding of unmodified phage was non-specific and was of considerably lower affinity than phage expressing the IQSPHFF motif (not shown).

To estimate the stoichiometry of NIRF binding to IQSPHFF phage, a known concentration of phage was incubated with VT680. The data showed that approximately 7.4±2.0 (SD) fluorochromes were bound to each phage. This number is within the range of p3 display peptides present on M13 phage. These results are consistent with binding of NIRF to IQSPHFF in a stoichiometry indicative of one NIRF per display peptide.

### Example 4

### Colorimetric Coated Slide Assay Using Peptide-NIRF Binding

To demonstrate the utility of IQSPHFF, a colorimetric immunoassay was performed.

Serial dilutions of VT680 (0.3M NaCO₃, pH 8.6) were spotted (1 μL) on an amino functionalized glass slide (Sigma-Aldrich) and were incubated overnight (4° C.), allowed to air dry, and then blocked with 5% BSA (0.3M NaCO₃, pH 8.6) for 1 hr at room temperature with gentle agitation. Following this, the slide was rinsed briefly with diphosphate buffered saline (DPBS) and incubated with 10¹² PFU of IQSPHFF displaying phage suspended in DPBS for 2-3 hrs. Supernatants were then discarded, and slide-bound phage were washed (3×5 min.) with DPBS plus 0.05% Tween-20. Next, anti-M13 antibody conjugated to horseradish peroxidase (HRP) was added to the slide (1:5,000+5% BSA in DPBS+0.05% Tween-20) and mixed with gentle agitation (45 min.). Following 4 washes (DPBS+0.05% Tween-20; final wash with distilled H₂O), the slide was developed by the addition of TMB substrate. Images of the slide were captured with a digital camera (Nikon CoolPix).

Dilutions of a benzindol fluorochrome NIRF (VT680) was spotted on chemically coated glass slides and incubated with IQSPHFF-displaying phage. FIG. 5B. VT680 binding to IQSPHFF was detected with anti-M13-HRP antibody. A concentration dependent binding of IQSPHFF-displaying phage to NIRF (VT680) was observed. A half-maximal response of 10 pmol (FIG. 3B) and a detection limit of 5 pmol of NIRF was calculated from the data. FIG. 5B. These data suggest that the IQSPHFF motif was selected to specifically bind to the NIRF (VT68), itself, and not non-specifically (i.e., for example, a chemical coupling comprising an interface between VT680 and bovine serum albumin; BSA).

### Example 5

### Peptide-NIRF Binding on Polystyrene Beads

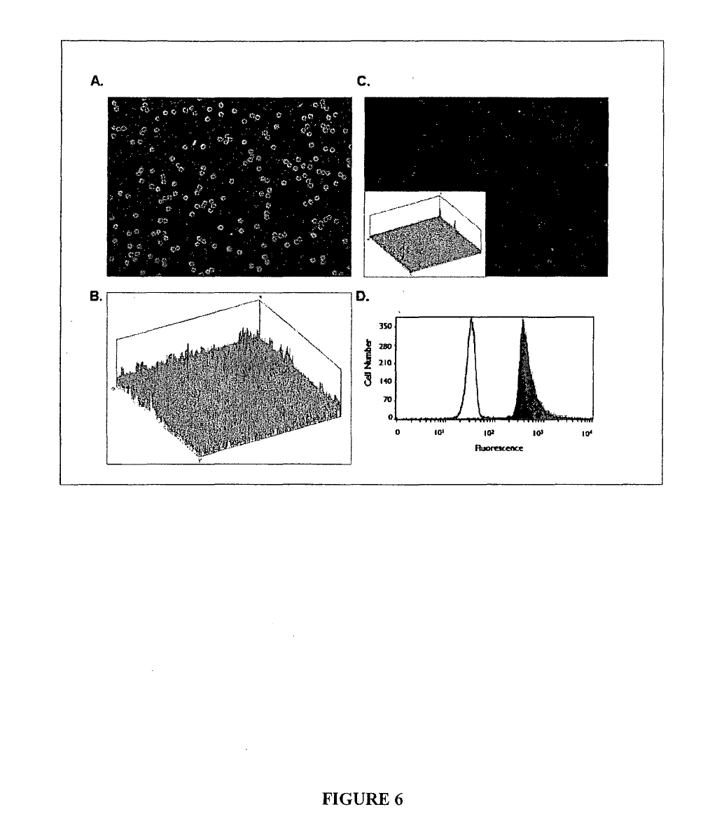

NIRF (VT680) was immobilized onto polystyrene microbeads and incubated with fluorescein isothiocyanate (FITC) labeled IQSPHFF-displaying phage. Fluorescence microscopy showed strong binding of IQSPHFF-phage to NIRF-modified microbeads but not to control microbeads. FIGS. 6A & 6B versus FIG. 6C and inset, respectively. Polystyrene beads conjugated to NHS-VT680 (NaHCO₃ buffer, pH 8.6) were blocked with 5% BSA, washed, and incubated with FITC-labeled IQSPHFF phage. VT680 labeling of beads was confirmed by FACS. Following incubation, beads were washed 5×, and supernatants containing unbound phage were discarded. Beads were then dissolved to approximately 5,000 per 4, plated in the wells of a 96-well tissue culture plate, and viewed by fluorescence microscopy using the 20× objective (Axiovert 100 TV, Zeus, Thornwood, N.Y.). Images with matched exposure times between sample and the negative control (blocked beads, no VT680) were collected via CCD camera. To obtain quantifiable fluorescence data from microscopic images, randomly selected bead images were analyzed for integrated pixel density (IPD) via Image J software.

Densitometric rendering confirmed a statistically significant difference using pixel topography renditions of FIG. 6A versus FIG. 6C (inset) wherein the average fold FITC intensity comparing NIRF-conjugated and unconjugated microbeads=6.1 (p<10⁻³⁶, t-test). Similar results were observed in reverse experiments when IQSPHFF-phage was bound to microbeads and challenged by soluble NIRF (data not shown). Monomeric soluble peptides were used to probe polystyrene microbeads covalently modified with NIRF. Flow cytometry results demonstrated NIRF-dependent binding of the soluble peptide. FIG. 6D.

For flow cytometry determinations, IQSPHFF phage (approximately 10¹² PFU.) were incubated with Bangs polystyrene beads (5×10⁶ in 1 mL 0.3M NaCO₃, pH 8.6, overnight at 4° C. A blocking step was then performed on the resuspended beads (5% BSA, 0.3M NaCO₃, pH 8.6, 1 hr). This suspension was pelleted and washed (3×1.0 mL DPBS+0.05% Tween-20 with brief vortexing), and then incubated for 1 hr with Genhance® 680 (0.1 mmol/L; 0.5 mL DPBS). Unbound Genhance® 680-containing supernatants were then discarded, and beads were washed again (4×1.0 mL DPBS+0.05% Tween-20) and suspended in DPBS. Bead samples (unlabeled, no phage+Genhance®, phage+Genhance®) were analyzed via flow cytometry (˜50,000 events counted). Statistically significant difference between emissions peaks from Genhance®+phage and Genhance® no phage samples was determined via Kolmogorov-Smirnov statistics.

To verify binding of free IQSPHFF peptide with NIRF (VT680) via FACS, 10⁶ polystyrene Bangs beads per reaction were labeled with VT680 (10¹⁰ fluorochromes per bead, 0.1 nmol per mL in 160 mL 0.3M NaCO₃, pH 8.6, 1 h@room temperature), washed (3×), blocked (5% BSA, 0.3M NaCO₃, pH 8.6, 1 hr.), washed again, resuspended in binding buffer (100 mL DPBS+0.05% Tween 20+1% BSA) overnight (4° C.) containing 1 mg of IQSPHFF peptide (+/−), washed (3×500 mL DPBS+0.05% Tween 20), probed with FITC-Streptavidin (1 hr. at RT X 1:200 in DPBS+0.05% Tween 20+1% BSA), washed (3×DPBS+0.05% Tween 20), resuspended in DPBS, and analyzed via FACS.

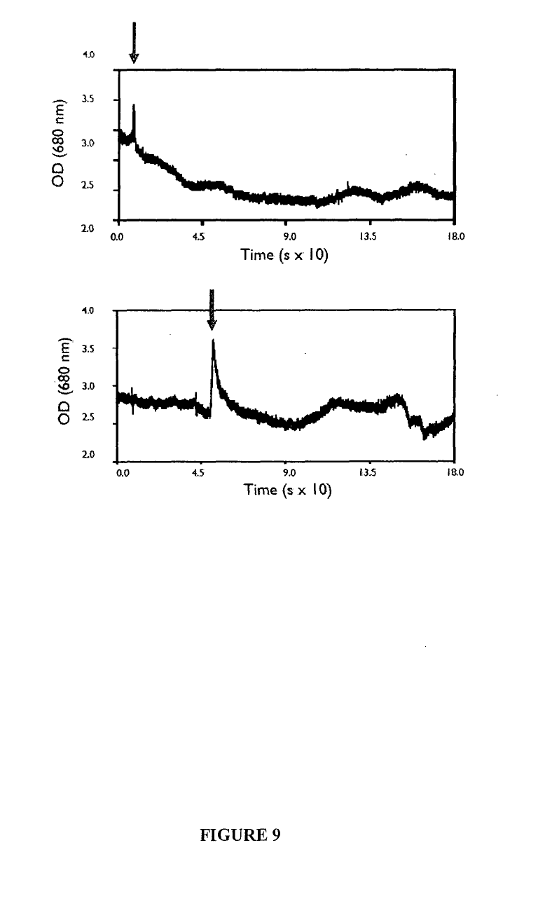

To further corroborate this result and to demonstrate the strength of the interaction, C18 reverse phase HPLC was used to determine whether IQ-tag—NIR complexes could form and co-elute. Free NIRF eluted immediately after the void volume of the column. In contrast, when peptide and NIRF were incubated then injected, the peak corresponding to fluorochrome shifted and was retained longer, indicating the binding of fluorochrome to peptide. FIG. 9.

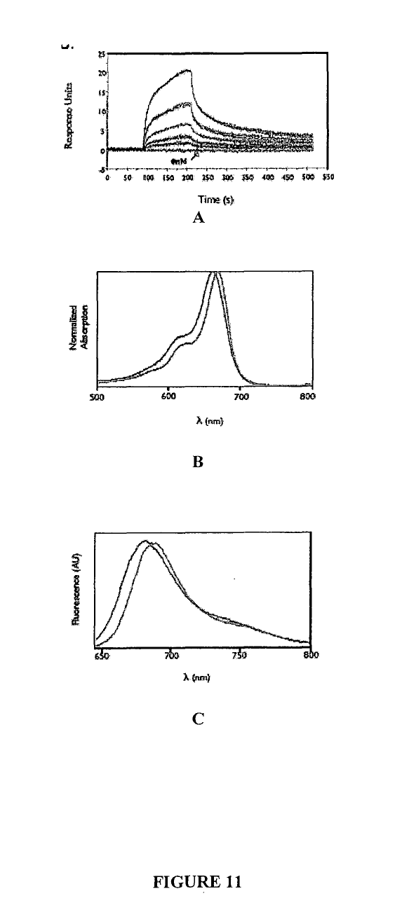

Surface plasmon resonance analysis showed a saturable, dose-dependent response with an affinity of the monomeric peptide binding to GH680 of approximately ˜100 nmol/L. FIG. 11A. NIRF fluorescence shift and relative quantum yield upon binding was also examined. Binding of IQ-tag to GH680 blue shifted the absorption by 14 nm and increased the relative quantum yield from 0.89 to 1, a 12% increase. FIGS. 11B and 11C.

### Example 6

### In Vivo Tumor Targeting

Peptide-NIRF interactions were used in vivo by a tumor-targeted phage labeled with NIRF.

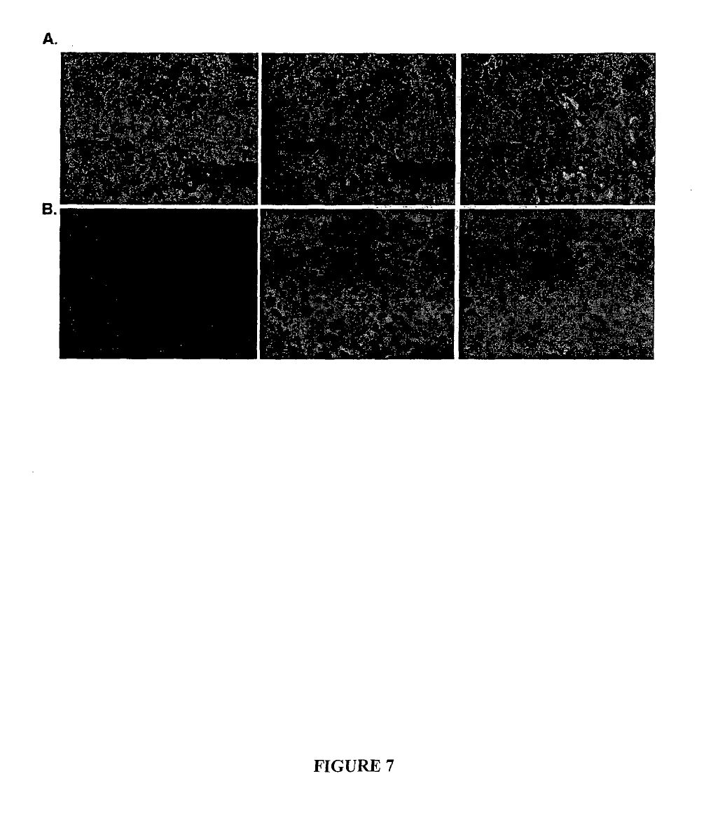

LLC bearing mice were injected intravenously (IV) with a NIRF-labeled SPARC-phage. Serial sections of tumors demonstrated high levels of SPARC-phage in tumors. FIG. 7A, red areas. When adjacent tumor sections were incubated with IQSPHFF-FITC labeled phage colocalization was observed. FIG. 7A, green and red/green areas. In contrast, data from negative control phages did not co-localize with differentially labeled IQSPHFF-FITC demonstrating IQSPHFF's specificity and utility. FIG. 7B.

### Example 7

### In Vitro and In Vivo Cell Imaging

This experiment determines the ability of the IQSPHFF peptide to image cells, both in vitro and in vivo.

### In Vitro Cell Labeling

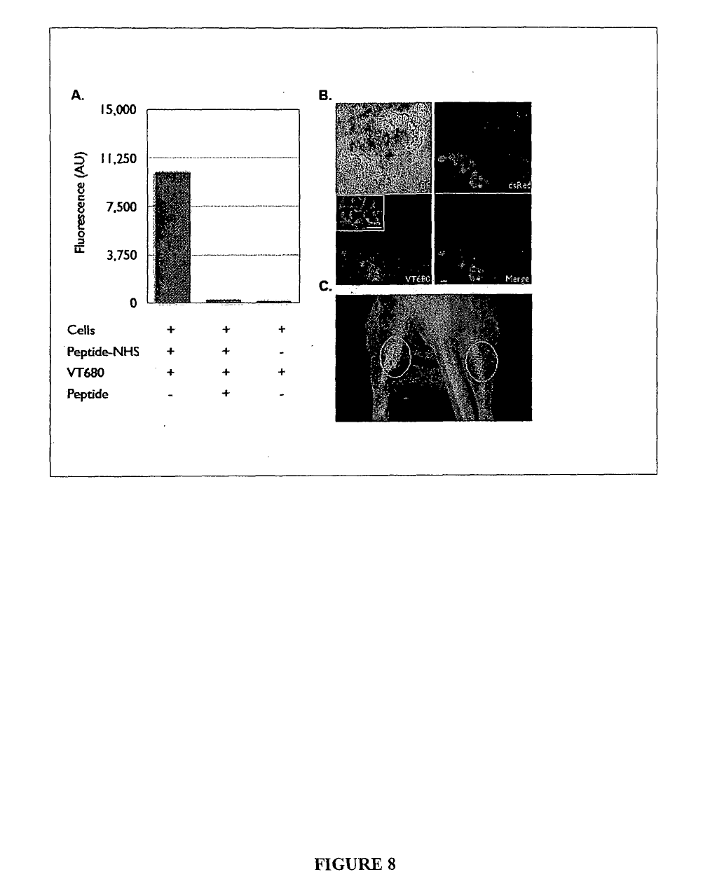

HT1080 colon adenocarcinoma cells (20,000 cells/well) were washed 3× with PBS to remove media then incubated with 0.1 mL of 100 mM of Ac-IQSPHFF-NHS peptide or vehicle (PBS with 1% DMSO) for 1 hour at 37° C. Subsequent to incubation, cells were washed then incubated for 1 hour at 37° C. in the dark with 1 nmole of GH680 or 1 nmole of GH680+5 nmoles of underivatized peptide (competition experiment). Cells were then washed 6× with PBS+0.1% Tween 20 and analyzed via fluorescence plate reader GeminiXS (Molecular Devices). The IQSPHFF peptide sequence was covalently conjugated onto HT1080 cells and incubated with 5 nM of NIRF (VT680). Cells conjugated with IQSPHFF bound VT680>120-fold better than cells not conjugated with IQSPHFF. In addition, free (unconjugated) IQSPHFF peptide was able to compete for 98% of the binding of fluorochrome to peptide labeled cells, indicating the specificity of the interaction. FIG. 8A.

One of the bioapplications where a peptide-NIRF binding pair may be useful is in protein and cell tracking. In a first transfection experiment, a construct was created with IQSPHFF fused to the amino terminus of the platelet-derived growth factor receptor (PDGFR) transmembrane domain and transfected into a tumor cell culture. Expression of the IQSPHFF-PDGFR fusion peptide sequence in combination with VT680 allowed the visualization of cells. An identical cell image was obtained using an PDGFR-red fluorescent protein (dsRed) fusion peptide sequence. FIG. 8B (compare lower left micrograph and upper right micrographs, respectively). A merged micrograph demonstrates co-localized expression, verifying that VT680 was binding to a cell surface membrane protein. VT680 did not bind to any cells without expression of the recombinant protein. (data not shown). These date, demonstrate the specificity of a high affinity peptide (i.e., for example, IQSPHFF) for a NIRF (i.e., for example, VT680) and the utility of such a NIRF-high affinity peptide binding pair for cell based imaging.

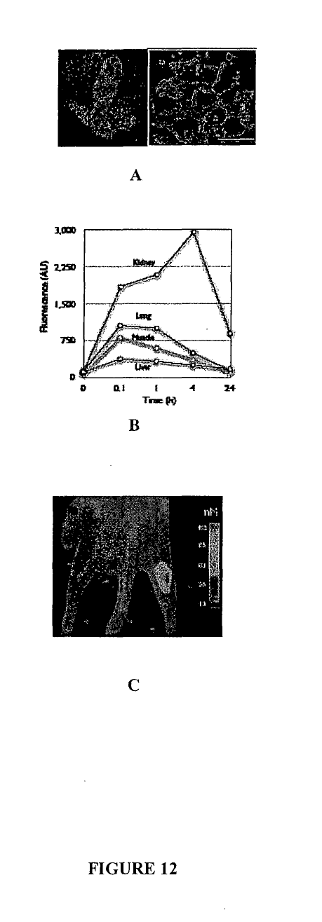

In a second transfection experiment, a bicistronic construct expressing dsRed and a fusion protein with IQ-tag fused to the amino terminus of the platelet-derived growth factor receptor (PDGFR) transmembrane domain was subsequently created to directly image protein expression and tumor cells. Transfection and expression of the peptide was confirmed by the co-expression of dsRed. HEK-293T cells that were expressing dsRed, also bound benzindolium fluorochrome. GH680 did not bind to cells devoid of dsRed expression. In addition, cells expressing dsRed were found to bind GH680 in the plasma membrane, as visualized by confocal microscopy. FIG. 12A.

### In Vivo Cell Labeling

The above bicistronic construct was further extended for in vivo visualization and tracking of cells and the PDGFR protein. Biodistribution experiments show that GH680 was rapidly eliminated via renal excretion with lung, muscle, and liver fluorescence minimal within 4 hours after injection. FIG. 12B. Importantly there was no non-specific binding to muscle tissue where titin (a protein with the nearest sequence to IQ-tag) is expressed abundantly. To determine whether cells could be imaged in vivo using this system, IQ-tag expressing cells were first implanted subcutaneously into nude mice, followed by a systemic injection of GH680 and imaging by fluorescence mediated tomography (FMT). FIG. 12C. After intravenously injecting GH680, IQ-tag expressing cells became brightly fluorescent with the signal persisting for over 24 hours whereas contralaterally implanted control cells showed negligible fluorescence (42.2 nM vs 1 nM in control leg; p<0.0001).

A second transfection experiment confirmed that vector DNA containing bicistronic mammalian expression cassettes encoding ds-Red and a PDGFR transmembrane domain chimera exposes the IQSPHFF peptide. For confocal microscopy imaging of HEK-293T cells, 100,000 cells per well of a 4-well chamber slide (Nunc, Rochester, N.Y.) were plated then transfected 16 hrs later using Lipofectamine2000 reagent at a concentration of 1.2 mg DNA and 2 ml of Lipofectamine per well, according to manufacturer's instructions. 68 h after transfection, wells were washed gently with DPBS and replaced with 200 ml of DPBS containing 50 mmol/L GH680 680 and 1% BSA for 1 h at 37 C. Treated wells were then washed (3×DPBS+1% BSA+0.01% Tween-20), fixed (5-10 min.×DPBS+2% paraformaldehyde), then imaged via the 20× objective of the Nikon Axiovert-100 inverted microscope and IPLab software and via the 40× objective of a Zeiss LSM Pascal confocal microscope (Carl Zeiss, Thornwood, N.Y.).

Animals were injected either, with or without, xenografts of tumor cells expressing the peptide-NIRF construct. Fluorescence is observed using FMT in mice only on limbs having tumors. FIG. 8C.

Serial tissue sections (˜5-10 mm diameter) of LLC xenograft tumors from animals that were injected with VT680-conjugated SPARC targeted phage were snap frozen or OCT embedded were obtained. Sections were then suspended in a fixing solution (5 min.×4% paraformaldehyde, PBS), rinsed in PBS, then blocked for 1 hour with 5% horse serum in a humidity chamber. 3×10¹⁰ FITC-conjugated phage (±IQSPHFF p3 display peptide) were added to the sections in 100 mL drops (1 hr. with humidity). Slides were then washed (PBS, 3×5 min.), and rinsed briefly with distilled H₂O, Slides were then mounted with Vectashield® (Vector Laboratories, Burlingame, Calif.). Images were captured under identical exposure/gain conditions using FITC and Cy 5.5 filters (Nikon Eclipse 20×).

### Example 8

### Fluorescence Activated Cell Sorting Detection of Fluorochrome High Affinity Binding to Cell Surface Peptide Expression

Cells expressing an IQSPHFF peptide sequence are washed with ice cold staining buffer (PBS/1% BSA/azide). Next, the cells are incubated for 30 minutes on ice with 10 micrograms/ml of VT680 fluorochrome conjugated to affinity purified rabbit anti-B305D polyclonal antibody. The cells are washed 3 times with staining buffer and then incubated with a 1:100 dilution of a goat anti-rabbit Ig (H+L)-FITC reagent (Southern Biotechnology) for 30 minutes on ice. Following 3 washes, the cells are resuspended in staining buffer containing prodium iodide, a vital stain that allows for identification of permeable cells, and analyzed by FACS. IQSPHFF surface expression was confirmed on tumor cells and that stably overexpress the cDNA for IQSPHFF.

### Example 9

### ELISA Determination of High Affinity Peptide/Fluorochrome Binding Pairs

The N-hydroxysuccinimide (NHS) ester of the benzindol fluorochromes VT680, GH680, Cy3.5, Cy5.5 or AF750 (1 mg) were conjugated to bovine serum albumin (BSA 100 mg/mL; 0.3 M NaCO₃ buffer, pH 8.6) under reaction conditions of approximately tenfold molar excess VT680 to BSA. BSA-fluorochrome conjugate or BSA was adsorbed (5.0 μg/0.1 ml well, w/vol) overnight to wells of a Nunc Maxisorp plage (Nunc, Rochester, N.Y.). Supernatants were discarded and washed twice with PBS and blocked (5% BSA, 0.3 M NaCO₃, pH 8.6, room temperature, 45 min). After two washes (PBS) to remove blocking buffer, 1:2 serial dilutions (DPBS+1% BSA) of phage solution (˜10¹¹ to ˜10⁹ PFU/well) were added to descending rows of plate wells and incubated for 1 hour. Subsequent to incubation, wells were washed (5×PBS+0.05% Tween-20) and incubated (1:5,000, DPBS+1% BSA) with anti-M13 conjugated to HRP (GE Biosciences, Piscataway, N.J.) for 45 min. Next, wells were washed again (5×PBS+0.05% Tween-20) and developed with tetramethylbenzidine (TMB), and absorbance at 650 nm was determined (Emax, Molecular Devices, Sunnyvale, Calif.).

Readout was depicted as the mean difference between absorbances in the BSA-fluorochrome containing wells and the BSA-only (negative control) wells. EC₅₀ values were obtained using sigmoidal dose response (variable slope) curve fitting from Prism 4 (GraphPad Software, San Diego, Calif.).

### Example 10

### Synthesis of a IQSPHFFGGSK Peptide

Peptide synthesis was performed on an automated solid phase peptide synthesizer (433A, Applied Biosystem, Foster City, Calif.) employing Fmoc methodology on Fmoc-Resin (154 mg, 0.1 mmol). Upon completion of the synthesis, the peptide-resin in DMF (4 mL) was acetylated using acetic anhydride (30 μL, 3.3 mmol) and diisopropylethylamine (DIPEA, 40 μL, 3.8 mmol). The resulting acetylated peptide resin was cleaved from the resin using 95% TFA/2.5% triisopropylsilane (TIS)/2.5% H₂O for 2 h, filtered to remove the resin, and precipitated in methyl-tert-butyl ether (MTBE). The precipitate was dissolved in DMF (4 mL), and reacted with N-hydroxysuccinimide (NHS, 58 mg, 0.5 mmol), dicyclohexylcarbodiimide (DCC, 103 mg, 0.5 mmol), and dimethylaminopyridine (DMAP, 61 mg, 0.5 mmol) for 1 h. The solution was filtered through cotton wool to remove dicyclohexylurea, and precipitated with MTBE (10 mL) to yield the crude product. Peptide mass was verified using mass spectroscopy (+ESI-MS (60 V, MeO H) m/z=1606.8 (MH+)).

### Example 11

### Stable Transfection And Labeling of HT1080

This example presents one embodiment of a method to construct a stable cell line capable of expressing a high affinity peptide suitable for in vivo cell tracking.

### Example 12

### In Vivo Cell Imaging

This example presents one embodiment of a method of injecting a vector comprising a high affinity peptide for in vivo transfection suitable for in vivo cell imaging.

For in vivo imaging of HEK-293T cells, 5 million cells were transfected into twin wells of a 6-well plate. IQ-tag-PDGFR transfected and mock transfected cells were injected locally for imaging and then probed via systemic injection of GH680 (2 moles). Imaging was performed by fluorescence mediated tomography (FMT). Mice were anesthetized by inhalation anesthesia (2% isoflurane, 1 L/min O2) using an isoflurane vaporizer (Braintree Scientific, Braintree, Mass.). FMT experiments were performed using a commercially available imaging system (VisEn Medical, Woburn, Mass.). Data sets were acquired at wavelengths 680/700 nm excitation/emission in anesthetized mice.

Image data sets were reconstructed using a normalized Born forward model adapted to small mouse models. Montet et al., “Tomographic fluorescence mapping of tumor targets” Cancer Res 65:6330-6336 (2005). Image acquisition time per animal was 2 minutes and reconstruction time was 1-2 minutes. Images were displayed as raw data sets (excitation, emission, masks) and as reconstructed 3D data sets in axial, sagittal and coronal planes. Fluorochrome concentration in the target was automatically calculated from reconstructed images and expressed as pmol fluorochrome/defined target volume. Biodistribution experiments were conducted after systemic administration of GH680 (2 nmoles) by removing organ samples and measuring fluorochrome concentrations at different time points.

### Example 13

### Affinity Determinations of Fluorochrome Binding Peptides

This example provide one method of determining the binding affinities of fluorochrome binding peptides. In brief, a phage display screening in accordance with Example 1 was utilized except that the libraries express random 15 amino acid fragments of all coat proteins.

### Example 14

### Cloning

To generate IQSPHFF peptide fused to the N-terminus of g7 splice overlap, extension PCR was performed using M13KE RFDNA according to manufacturer's instructions (New England Biolabs, Beverly, Mass.). p7-IQSPHFF-containing phage clones were positively screened by mobility shift in PCR and confirmed by DNA sequencing.

### Example 15

### Creation of IQ-Tag Transgenic Mice

A fragment of the Cre lox promoter is ligated into a plasmid PDX-1 (R1m) RGBpBS-KS containing the entire coding sequence of rat PDX-1. The recombinant plasmid also contains an IQ-Tag sequence (i.e, for example, SEQ ID NO: 1) between the PDX-1 cDNA and the promoter and the 0.52-kb rabbit β-globin polyadenylation signal cassette 3′ to the PDX-1 cDNA. DNA sequencing and polymerase chain reaction (PCR) analyses confirms the proper orientation of the PDX-1 cDNA.

Transgenic (TG) mice are produced by following standard transgenic protocols. DNA is isolated from tail snips obtained from mice age 4 weeks. Tails are digested with proteinase K (Boehringer Mannheim, Indianapolis, Ind.) and the DNA precipitated with ethanol. Genomic DNA is analyzed by PCR using primers specific for the detection of the transgene (rabbit β-globin primer RBG 5′ CTCTGCTAACCATGTTCATGCCT 3′ and the PDX-1-specific reverse primer 5′ GCAGGCCAGCCAGGCTACAAAAAT 3′). The endogenous gene is amplified with an IQ-Tag forward primer and the same PDX-1 reverse primer. A total of 12 founders are identified, and a subset of these are characterized morphologically. Several lines are bred to homozygosity, as confirmed by backcrossing to FVB wild-type mice. After the initial characterization, subsequent analysis was performed in the representative homozygous IQ-tag line.

1. A peptide comprising a high affinity binding site to a fluorochrome.

2. The peptide of claim 1, wherein said high affinity binding site comprises a submicromolar affinity to said fluorochrome.

3. The peptide of claim 1, wherein said high affinity binding site comprises an affinity of less than 0.1 nanomolar to said fluorochrome.

4. The peptide of claim 1, wherein said high affinity binding site comprises an affinity of approximately between 10-100 picomolar to said fluorochrome.

5. The peptide of claim 1, wherein said high affinity binding site comprises an affinity of approximately between approximately 1-10 picomolar to said fluorochrome.

6. The peptide of claim 1, wherein said high affinity binding site comprises between approximately four to fifteen amino acids.

7. The peptide of claim 6, wherein said binding site comprises I, S, and at least two F's.

8. The peptide of claim 7, wherein said at least two F's are consecutive.

9. The peptide of claim 7, wherein said binding site further comprises Q.

10. The peptide of claim 7, wherein said binding site further comprises P.

11. The peptide of claim 10, wherein said binding site further comprises H.

12. The peptide of claim 11, wherein said P and said H are consecutive.

13. The peptide of claim 1, wherein said binding site comprises IQSPHFF.

14. The peptide of claim 1, wherein said binding site comprises IQSPHFFGGSK.

15. The peptide of claim 1, wherein said fluorochrome is selected from the group consisting of VT680, GH680, AF750, Cy3.5, and Cy5.5.

16. The peptide of claim 1, wherein said peptide is part of a fusion protein.

17. The peptide of claim 16, wherein said fusion protein further comprises a cell targeting moiety.

18. The peptide of claim 17, wherein said cell targeting moiety comprises platelet derived growth factor receptor.

19. The peptide of claim 1, wherein said fluorochrome comprises a benzindol fluorochrome.

20-63. (canceled)
